(19) United States
(12) Patent Application Publication
(10) Pub. No.: US 2023/0215567 A1
(43) Pub. Date: Jul. 6, 2023

(54) COVIDOMETER, SYSTEMS AND METHODS TO DETECT NEW MUTATED COVID VARIANTS

(71) Applicant: Dmytro Kviatkovskyi, Surrey (GB)

(72) Inventor: Dmytro Kviatkovskyi, Surrey (GB)

(21) Appl. No.: 17/975,105

(22) Filed: Oct. 27, 2022

Related U.S. Application Data
(60) Provisional application No. 63/294,909, filed on Dec. 30, 2021.

(51) Int. Cl. G16H 50/20; G01N 33/569; G16H 50/50

(52) U.S. Cl. CPC G16H 50/20; G01N 33/56983; G16H 50/50; G01N 2333/165; G01N 2469/20

(57) ABSTRACT

Methods, systems and devices for determining COVID disease, including receiving symptom data values, calculating first differentials (positive or negative) for the first virus strain by comparing the values to first predetermined symptom threshold values for the first virus strain, using the first differentials to detect a second virus strain with a mutated virus genome code based on a correspondence of its symptoms to the first differentials, calculating second differentials (positive or negative) for the second virus strain by comparing the values to second predetermined symptom threshold values for the second virus strain, creating a superset of the first and second differentials, detecting correlations within the superset, determining that the person has the first or second virus strain when at least one detected correlation indicates that the person has contracted the first or second virus strain, outputting a result indicating a presence or absence of COVID in a person.

### BACKGROUND OF THE INVENTION

### Field of the Invention

The present invention relates to diagnosing an individual's health, and more particularly, to systems and methods for detecting viral infections, e.g., new mutated COVID variants.

### Description of the Related Art

Coronavirus disease 2019 or COVID-19 (SARS-CoV-2) and the many thousands of COVID variants have spread worldwide, leading to an ongoing global pandemic. COVID-19 cannot be distinguished from other acute respiratory infections, such as the common cold or other viral infections, by symptoms alone. COVID-induced pneumonia also can be clinically indistinguishable from pneumonia caused by other pathogens. The COVID pandemic has swept the entire world, becoming a fundamental global tragedy, as it is virtually impossible to isolate oneself from the virus.

Precious human lives are lost every day and the COVID pandemic has swept away millions of people from around the globe. Over the entire time of the pandemic, more than 270 million people in the world have been infected with COVID-19. As of September 2022, more than 6.5 million people worldwide have died from or with the infection. Many cases of COVID diseases and deaths are not included in the official statistics. There is no specific, effective treatment or cure for the COVID virus. In 2020 highly effective vaccines were introduced and are beginning to slow the spread of the COVID-19 virus strain or new mutated COVID variants. However, for those who do not have a vaccination, as well as for the estimated millions of immunocompromised persons who are unlikely to respond robustly to vaccination, timely diagnosis and treatment remain very important.

Viruses tend to mutate, and COVID-19 is no exception. As a consequence, severe acute respiratory syndrome coronavirus SARS-CoV-2, the virus that causes COVID-19, has many variants. COVID-19 is an RNA virus, i.e., it has molecules of ribonucleic acid (RNA) as its genetic material. Each time the virus copies itself, the RNA sequence may change, which causes mutations. The virus' traits, such as its contagiousness and lethality, also change. Currently, there are thousands of coronavirus strains. Some are believed, or have been believed, to be of particular importance due to their potential for increased transmissibility, increased virulence, or reduced effectiveness of vaccines against them.

At the end of November 2021, another dangerous variant emerged—B.1.1.529, which received the name Omicron and it has been assessed as highly dangerous by the World Health Organization. The Omicron variant has been found to better evade antibodies than the Delta variant, which is now the most widespread COVID variant in the world. The Omicron variant has more than 50 mutations from the original COVID-19 virus strain, and most mutations are in the gene encoding the spike protein, which is the target of most vaccines.

The WHO supposes that, after a few mutations, all current diagnostic means, vaccines, and coronavirus drugs may become ineffective against the Omicron variant. Besides, this variant spreads faster than the previous ones, and it may affect the whole global population, including those who have already recovered after being infected by another COVID variant. In August 2022, a new Deltacron variant, which is a hybrid of variants Delta and Omicron, was first detected in Russia.

Thus, new and mutated COVID variants have sprouted up all around the world. These new variants have a different chromosomal genome structure, behave differently, and affect different vital organs in the human body, such as the new Brazilian variant or new Centaurus variant, which were first detected in July 2022. Therefore, we are facing a unique and tragic reality, in which COVID-19 is constantly mutating, spawning new autonomous variants that may be even more hazardous for people than the original variant.

At the same time, the system of medical healthcare delivery in the world is facing a critical point in its evolution. While many COVID variants have vaccines, they do not exist in quantities that might be needed and may not be located where an outbreak of a new COVID variant was to occur. Though lockdown was an effective tool, it cannot be implemented for a longer duration if every country and its economy is to function normally.

A number of existing drawbacks of the modern medical practice can be overcome using the proposed systems and methods for detecting new COVID variants of the claimed invention that enable a comprehensive diagnosis of viral infections. Timely detection of new COVID variants and their effective treatment is possible only if a viral disease is diagnosed comprehensively and systematically, using methods for detecting new COVID variants. Therefore, there remains a need to develop detection and diagnostics modern systems for COVID-19 and new COVID variants.

It is therefore proposed to add new systems and methods for detecting new COVID variants to conventional methods, wherein a viral disease is diagnosed and analyzed based on the patient's individual physiological parameters. This would enable us to calculate individual risks of infection for the patient and determine a possible course of the disease, i.e., if the patient has a required level of antibodies (immunity) against a previous COVID variant, but is infected by a new COVID variant with a mutated genome.

### SUMMARY OF THE INVENTION

Sensors collect biochemical and biophysical data from a person representing their symptoms and are either connected to the person or perform data collection remotely. Sensors collect the symptom data values from the person for detecting respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

Also, the symptom data values may be obtained at medical institutions that run laboratory medical tests (e.g., antigen test, molecular test, antibody test) and laboratory medical examinations. Laboratory medical tests include the reverse transcription polymerase chain reaction (RT-PCR) test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay. Laboratory medical examinations include chest CT scans, checking for elevated body temperature, checking for low blood oxygen levels, etc.

The sensors may include a smartphone, pulse oximeter, body temperature thermometer, heart pulse sensor, heart monitor, electrodermal activity (EDA) sensor, respiratory sensor, etc. The sensors may also include biosensors that are analytical devices that combine a biological component (e.g., tissue, microorganisms, organelles, cell receptors, enzymes, antibodies, nucleic acids, etc.) with a physicochemical detector.

Types of bio sensors of present invention include those that use enzymes as a biologically responsive material, whole cell metabolism, ligan debinding and antibody-antigen reaction. The types of person biological information that can be used for collection of the symptom data values can include but is not limited to, DNA, antibodies, whole blood, blood plasma, serum samples, nasopharyngeal swab, throat swabs, deep airway material, saliva, any bodily fluid, thyroid function, genetic testing, skin testing, etc.

The server is connected to sensors and biosensors via a data exchange system, collecting symptom data values, which includes antibody level, heartrate, blood pressure, pulse oxygen level, respiratory rhythm/rate, etc., and has a network connection to a person's user device. Sensors and biosensors may be in communication with a smartphone which, in turn, is in communication with at least one computing device via an Internet connection. The computing devices can be of different types, such as a PC, laptop, tablet, smartphone, smartwatch, etc.

The process may be performed by the server or processor in conjunction with the user device (e.g., running a software program provided by the server or processor). The data is transferred by the sensor using a secure encoded channel. Also the symptom data values can be collected through, manual input into the system, wireless computer protocols, LIS servers, HL7 diagnostic protocols, HIPAA compliant database queries, batch processing from medical records, any other means of digital entry, etc.

All symptom data values collected from person using sensors, biosensors, medical tests, examinations are combined into a consolidated database on the server. The obtained data values may provide direct evidence the person is experiencing one of the symptoms of COVID disease. The server may operate in a networked environment using logical connections to one or more remote computers. The remote computer (or computers) may be another personal computer, a server, a router, a network PC, a peer device, or other common network node.

The server is connected with a central site central processing unit and comprises a database of the symptom data values received and a database of predetermined symptom threshold values for all COVID variants (SARS-CoV-2 virus strains): original COVID-19 (SARS-CoV-2), Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc.

A computer implemented software application (or a non-transitory computer-readable medium that stores the instructions) is downloadable to the server and is executed by a processor in order to induce the system to perform the algorithm of present invention for determining COVID disease in a person. The algorithm may be implemented on the computer (or another smart device, such as a smartphone, tablet, or laptop) or other software (Cloud server). When implemented on the smartphone, the algorithm may be a component of the application. When implemented on a computer, the algorithm may be a component of a non-transitory computer-readable medium (removable storage drive, a hard disk installed in a hard disk drive, flash memories, removable discs, non-removable discs, etc.) storing a program of instruction.

The algorithm comprises the steps of: receiving a plurality of symptom data values from a person, calculating the differentials by comparing the received values to predetermined symptom threshold values for the original COVID-19 virus strain (the first SARS-CoV-2 virus strain), using the differentials to detect a COVID variant (the second SARS-CoV-2 virus strain) that is closely related to the disease based on a correspondence of its symptoms to the differentials, calculating the differentials by comparing the received values to predetermined symptom threshold values for a closely related COVID variant (the second SARS-CoV-2 virus strain), creating a set of all differentials, analyzing the set of differentials to detect correlations within the set indicative of relationships between the differentials, determining if the person has the first SARS-CoV-2 virus strain or the second SARS-CoV-2 virus strain when at least one detected correlation indicates the person has contracted the first or second virus strain, outputting a result indicating a presence or absence of COVID disease in a person.

The calculated differentials are both positive or negative. The differentials are negative when the received symptom data values do not exceed the predetermined symptom threshold values, and positive when the received symptom data values exceed the predetermined symptom threshold values. Therefore, the set of differentials may contain positive and negative differentials, and the further analysis of the set of differentials is carried out to detect correlations between the positive and negative differentials within the set.

In an aspect, the closely related COVID variant (the second SARS-CoV-2 virus strain) is detected such that its symptoms correspond to a majority of the differential detected by comparing the received values to predetermined symptom threshold values for the original COVID-19 virus strain (the first SARS-CoV-2 virus strain). In another aspect, the closely related COVID variant (the second SARS-CoV-2 virus strain) is detected such that its symptoms correspond to a minority of the differential detected by comparing the received values to predetermined symptom threshold values for the original COVID-19 virus strain (the first SARS-CoV-2 virus strain).

The created set of differentials is analyzed by using combinatorial data analysis, cluster analysis, regression analysis to detect correlations that are the mathematical or logical relationships between the values within the set. The combinatorial data analysis uses the order of the differentials within the set of differentials to define different combinations of the differentials and their correlations with each other. The cluster analysis uses the differences in the differentials within the set of differentials to define multiple groups of the differentials and to find correlations in each group. The regression analysis uses the constructing a network of curves within the differentials of the set of differentials such that its characteristic figures show correlations between the differentials.

A person skilled in the relevant art will recognize other steps may be applied for implementing the algorithm of the present invention. Thus, in an aspect, an algorithm further comprises the step of combining the differentials within the set of differentials into multiple groups based on the differences in the differentials. The differentials that were not included in the groups are not taken into account in the further analysis of the set of differentials. Based on the detected multiple groups of differentials, the set of differentials is analyzed to detect correlations indicative of relationships between the groups of the differentials.

In another aspect, an algorithm further comprises the step of detecting yet another COVID variant (the third SARS-CoV-2 virus strain) based on a correspondence of its symptoms to the differentials calculated by comparing the received values to predetermined symptom threshold values for the closely related COVID variant (the second SARS-CoV-2 virus strain). Then differentials (positive or negative) are calculated between the received values and the predetermined symptom threshold values for yet another COVID variant (the third SARS-CoV-2 virus strain) for further creating the set of all differentials.

In an aspect, the yet another COVID variant (the third SARS-CoV-2 virus strain) is detected such that its symptoms correspond to a majority of the differential detected by comparing the received values to predetermined symptom threshold values for the closely related COVID variant (the second SARS-CoV-2 virus strain). In another aspect, the yet another COVID variant (the third SARS-CoV-2 virus strain) is detected such that its symptoms correspond to a minority of the differential detected by comparing the received values to predetermined symptom threshold values for the closely related COVID variant (the second SARS-CoV-2 virus strain).

In yet another aspect, an algorithm further comprises the step of analyzing the detected correlations using cluster analysis to define the same or similar correlations and combining these correlations into a group. The correlations that were not included in the groups are not taken into account in the further determination of COVID disease in a person. Based on the multiple groups of correlations detected, a COVID disease is diagnosed in the event that at least one detected group of correlations indicates the person is likely to have contracted the COVID disease.

Then in response to the determination, a result indicating a presence or absence of COVID disease in a person is outputting. In an aspect, the outputted result is storied to a database on a computer for further outputting or displaying. In another aspect, the outputted result is transmitted to a mobile electronic device using end-to-end encryption. In yet another aspect, the outputted result is loaded to a Cloud server shared by multiple computers.

This system of present invention can be implemented in the device of the “Covidometer” that determines if a person has a viral disease of an original COVID-19 virus strain or new mutated COVID variant and requires no lab work. The “Covidometer” is the small, portable battery powered device with computing resources in the form of a storage for biological sample (e.g., a box for biological materials), processor, memory, analyzer, screen, and it can determine at home whether a person has contracted the COVID disease.

The “Covidometer” is configured to cooperate with the biological component (e.g., the blood or airway material) by using sensors, biosensors and biochip. The whole sampling process is carried out by biosensors or a biochip that produce a signal (e.g., electrochemical change to detect presence of antigens) detectable by the sensors (e.g., an electrochemical immunosensors capable of distinguishing IgM and IgG antibodies from each other).

The “Covidometer” of present invention comprises a plurality of biosensors (or a biochip) that collect biological sample from the person, a plurality of sensors that gather values of data from the biological sample, a transmission system to transmit data from the biosensors (or the biochip) and sensors, a processor in communication with the transmission system to collect the data from the biosensors (or biochip) and sensors, a storage that receives and stores collected biological sample, a server in communication with the processor that comprise a database of the gathered values of the data and a database of predetermined symptom threshold values for all COVID variants (SARS-CoV-2 virus strains), a software application that is downloadable to and executable by the processor to cause the system to perform the steps of algorithm noted above, as well as means (e.g., a display monitor or printer) for outputting a result indicating a presence or absence of COVID disease in a person.

These and other systems, methods, objects, features, and advantages of the present invention will be apparent to those skilled in the art from the following detailed description of the preferred embodiment and the drawings.

Additional features and advantages of the invention will be set forth in the description that follows, and in part will be apparent from the description, or may be learned by practice using the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed. All documents mentioned herein are hereby incorporated in their entirety by reference.

### BRIEF DESCRIPTION OF THE ATTACHED FIGURES

The accompanying drawings, which are included to provide a further understanding of the invention and are incorporated in and constitute a part of this specification, illustrate embodiments of the invention and, together with the description, serve to explain the principles of the invention.

In the drawings:

FIG. 1 is a diagram illustrating an example of a system for detecting COVID variants.

FIG. 2 is a diagram illustrating another example of a system for detecting COVID variants.

FIG. 3 is a flowchart illustrating the steps of the algorithm for detecting COVID variants according to a first embodiment of the invention.

FIG. 4 is a flowchart illustrating the steps of the algorithm for detecting COVID variants according to a second embodiment of the invention.

FIG. 5 is a flowchart illustrating the steps of the algorithm for detecting COVID variants according to a third embodiment of the invention.

FIG. 6 is a flowchart illustrating the steps of the algorithm for detecting COVID variants according to a fourth embodiment of the invention.

FIG. 7 is a diagram illustrating the analysis of medical data of the invention.

FIG. 8 illustrates the table of differentials for COVID variants of FIG. 7.

FIG. 9 is a diagram illustrating hardware components of the system for implementing the method of combinatorial statistical analysis of FIG. 7.

FIG. 10 illustrates an example of the result of using the method of combinatorial statistical analysis of FIG. 7.

FIG. 11 is a diagram illustrating hardware components of the system for implementing the mathematical method of dense network of curves of FIG. 7.

FIG. 12 illustrates a graphical example of using the mathematical method of dense network of curves of FIG. 7.

FIG. 13 is a diagram illustrating an example of detecting COVID variants using medical testing.

FIG. 14 is a diagram illustrating another example of detecting COVID variants using medical testing.

FIG. 15 is a diagram illustrating hardware and software components for implementing the invention.

FIG. 16 illustrates components of the data merging module of FIG. 15.

FIG. 17 is a diagram illustrating an example of a computer system for implementing the invention.

FIG. 18 is a diagram illustrating another example of a computer system for implementing the invention.

FIG. 19 is a diagram illustrating an example of a system for detecting COVID variants implemented in the device of the “Covidometer.”

FIG. 20 is a diagram illustrating hardware components of the device of the “Covidometer.”

FIG. 21 is a diagram illustrating an example of the operation of the device of the “Covidometer.”

### DETAILED DESCRIPTION OF THE PREFERRED EMBODIMENTS

Reference will now be made in detail to the preferred embodiments of the present invention, examples of which are illustrated in the accompanying drawings.

The invention relates to systems and methods for detecting, analyzing, and diagnosing new COVID variants. Below are the main terms used in the present invention.

A virus is an infectious agent that replicates inside the living cells of an organism and infects all life forms, from plants and animals to humans. Examples of common human diseases caused by viruses include the common cold, influenza, chickenpox, and cold sores. Many serious diseases such as rabies, Ebola virus disease, AIDS (HIV), avian influenza, and SARS are caused by viruses. Viruses spread in many ways. Many viruses, including influenza viruses, SARS-CoV-2, chickenpox, smallpox, and measles, spread in the air by coughing and sneezing.

Coronaviruses are a group of related RNA viruses that cause severe acute respiratory syndrome diseases.

COVID-19 is a viral disease caused by severe acute respiratory syndrome coronavirus 2 (SARS-CoV-2).

The original COVID-19 is the original SARS-CoV-2 virus strain, which is the base for new COVID variants having a changed (mutated) virus genome code.

A COVID variant is a new mutated COVID variant that differs from the original COVID-19 (SARS-CoV-2) virus strain by having a changed (mutated) virus genome code.

A closely related COVID variant to the disease is a COVID variant that is closest in its symptoms to the person's diagnosed viral disease.

Major COVID variants include the COVID variants of concern (VOC) currently recognized by the World Health Organization, COVID variants of interest (VOI) which are or were recognized by the World Health Organization, other notable COVID variants.

COVID variants of concern—Alpha (lineage B.1.1.7), B.1.1.7 with E484K, Beta (lineage B.1.351), Gamma (lineage P.1), Delta (lineage B.1.617.2).

COVID variants of interest—Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529).

Other notable COVID variants—Lineage B.1.1.207, Lineage B.1.1.317, Lineage B.1.616, Lineage B.1.618, Brazilian variant, Centaurus variant, Deltacron variant.

Predetermined symptom threshold values for COVID variants are predetermined actual limits for specific symptoms provided in medical literature, which, when exceeded, show that the person has been infected by a COVID disease.

Medical guidelines are well established, documented in medical literature, and famous scientific facts, which indicate predetermined symptom threshold values for COVID variants.

A differential is a positive or negative difference between the values of the patient's biochemical and biophysical data obtained and predetermined symptom threshold values for COVID variants. According to conventional medical practice, the difference is calculated only when the data obtained from a sensor exceeds the threshold of a given symptom, and therefore, this difference can only be positive. A differential, on the other hand, is calculated even if the threshold has not been exceeded, and therefore it can be negative. (That is why the present invention uses differentials rather than differences.)

Correlation is any mathematical or logical relationship (dependence) between two random variables.

Sensors include the devices which collect the patient's biochemical and biophysical data for detecting respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

Sensors include biosensors. A biosensor is an analytical device which converts a biological response into an electrical signal and combines a biological component with a physicochemical detector. The sensitive biological element, e.g., tissue, microorganisms, organelles, cell receptors, enzymes, antibodies, nucleic acids, etc., is a biologically derived material or biomimetic component that interacts with, binds with, or recognizes the analyte under study.

Laboratory medical tests include the reverse transcription polymerase chain reaction (RT-PCR) test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay, etc.

Laboratory medical examinations include chest CT scans, checking for elevated body temperature, checking for low blood oxygen levels, etc.

The patient's individual data include the patient's individual physiological parameters, the patient's diseases that accompany COVID-19, and additional patient data.

Individual physiological parameters include parameters that describe the physiology of a given human organism, such as sex, height, weight, age, gender, blood type, blood sugar, blood pressure, immunity, etc.

Diseases that accompany COVID-19 include the patient's diseases that may accompany COVID-19 virus, such as tuberculosis, diabetes, severe immunosuppression, lymphoma, oncological diseases, ulcers, cardiovascular pathologies, nervous diseases, etc.

Additional patient data includes information regarding the ethnicity, area of living, quarantine stay length, vaccination history, social connections, etc.

The post-COVID syndrome is post-acute sequelae of COVID disease characterized by long-term sequelae appearing or persisting after the typical convalescence period of COVID disease. The post-COVID syndrome may cause symptoms that won't correlate with the diagnosis. For instance, so-called “COVID toes,” i.e., false chilblains of toes or fingers in smokers or people with cardiovascular pathologies, relate to the post-COVID syndrome. Also, researchers are reporting new-onset diabetes in COVID-19 patients, etc.

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural and logical changes may be made without departing from the spirit and scope of the present invention. The following detailed description is, therefore, not to be taken in a limiting sense, and the scope of the present invention is defined by the appended claims and their equivalents.

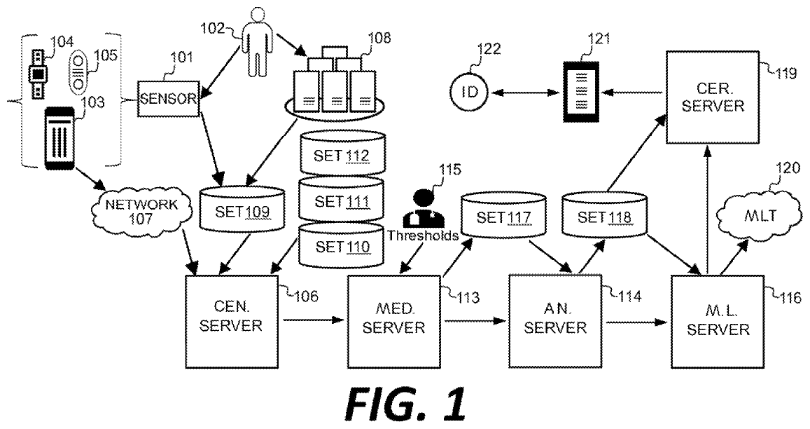

FIG. 1 is a diagram illustrating an example of a system for detecting COVID variants of the present invention. Sensors (including bio sensors) 101 collect biochemical and biophysical data from a patient 102. The sensors 101 are either connected to the patient or perform data collection remotely, and may include a smartphone 103, a pulse oximeter 104, a body temperature thermometer 105, etc., which send the data collected via a secure and encoded channel to a central server 106.

The method includes using a pulse oximeter 104 to acquire at least the pulse and blood oxygen saturation percentage, which is transmitted wirelessly to a smartphone. The body temperature thermometer 105 may be any suitable device configured to sense the body temperature and output information indicative of the body temperature. The body temperature thermometer 105 may output information indicative of the body temperature to the user device 103 (e.g., smartphone), for example, via direct, short-range, wireless communication signals (e.g., Bluetooth), via the local area network, etc.

Also, sensors 101 may include a heart pulse sensor, a heart monitor, an electrodermal activity (EDA) sensor, a respiratory sensor, etc. For example, data indicative of the heart activity of the patient may be received, for example, from the heart pulse sensor. Heartrate variability may be determined, for example, based on data received from the heart monitor. Sensors 101 may be in communication with a smartphone 103, which, in turn, is in communication with at least one computing device via a wide area network 107 (WAN), such as the Internet. The computing devices can be of different types, such as a PC, laptop, tablet, smartphone, smartwatch, etc., using one, or different operating systems or platforms.

The sensors 101 may include biosensors. The biosensors are an analytical devices, used for the detection of a chemical substance, that combine a biological component with a physicochemical detector. Biosensors are capable of converting the biological response into an electrical signal. The sensitive biological element, e.g., tissue, microorganisms, organelles, cell receptors, enzymes, antibodies, nucleic acids, etc., is a biologically derived material or biomimetic component that interacts with, binds with, or recognizes the analyte under study.

Types of biosensors 101 of present invention include those that use enzymes as a biologically responsive material, whole cell metabolism, ligan debinding and antibody-antigen reaction. In an enzyme response system, a biocatalytic membrane accomplishes conversion of a reactant to a product. This reaction is determined and sensed by a transducer which converts it to an electrical signal. The transducer makes use of a change accompanying the reaction such as heat output (or absorption) by the reaction (calorimetric biosensors), changes in distribution of charges causing an electrical potential to be produced (potentiometric biosensors), movement of electrons produced in a reduction oxidation reaction (amperometric biosensors), light output during the reaction or a light absorbance difference between the reactants and products (optical biosensors), or effects due to the mass of the reactants or products (piezo-electric biosensors).

The central server 106 is connected to the sensors 101 via a data exchange system, collecting biochemical and biophysical data, which includes heart rate, blood pressure, pulse oxygen level, respiratory rhythm/rate, etc. The central server 106 has a network connection to a patient's user device (e.g., a smartphone 103) and is connected to the wide area network 107. The system may also be configured to periodically or continuously monitor the health of the patient 102 (e.g., at least once per day).

It should be appreciated that all data may be acquired manually (e.g., by requiring the patient to enter the information), including respiratory rate (e.g., number of breaths per minute), body temperature, and blood pressure (e.g., systolic pressure, diastolic pressure), and used together with other values, such as Perfusion Index (PI %), Perfusion Index Trend Waveform, age, weight, sex, etc., to determine whether the patient 102 is suffering from a major COVID variant. The recording of the data is preferably done through the smartphone 103, or an application operating thereon, using a simple user interface. Alternatively, the process may be performed by the central server 106 in conjunction with the user device (smartphone) 103 (e.g., running a software program provided by the central server 106).

The biochemical and biophysical data 108 may be obtained at a medical institution that runs laboratory medical tests for COVID disease (e.g., antigen test, molecular test, antibody test) and laboratory medical examinations for COVID disease. Laboratory medical tests include the reverse transcription polymerase chain reaction (RT-PCR) test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay, etc. Laboratory medical examinations include chest CT scans, checking for elevated body temperature, checking for low blood oxygen levels, etc. Also, biochemical and biophysical data 108 may be obtained by the patient 102 themselves at home, either manually or automatically.

A set 109 comprises decrypted biochemical and biophysical data that has been obtained from sensors 101 and through laboratory medical tests, laboratory medical examinations 108. A set 110 comprises the patient's individual physiological parameters, including their sex, age, height, weight, blood type, blood sugar, blood pressure, immunity, vaccination history, etc. A set 111 comprises patient's diseases that accompany COVID-19, such as tuberculosis, diabetes, pregnancy, severe immunosuppression, lymphoma, oncological diseases, ulcers, cardiovascular pathologies, nervous diseases, etc. A set 112 comprises additional patient data, such as ethnicity, area of living, quarantine stay length, social connections, etc.

The patient's individual physiological parameters, any patient diseases that accompany COVID-19, and additional patient data are inputted into the system by the patient 102 themselves, or by a doctor using a smartphone 103 interface. All these sets are stored on the central server 106, which is connected to a medical server 113 and an analytical server 114, in datasets that are sent to the medical server 113 and the analytical server 114.

Separately, a database with predetermined symptom threshold values 115 for major COVID variants is generated on the medical server 113. For this, medical guidelines containing up-to-date medical information for definition of predetermined symptom threshold values 115 for all major COVID variants, or the listing of the predetermined symptom threshold values 115 for all major COVID variants are uploaded to the medical server 113. By relying on well-established, medically documented, famous scientific facts, predetermined symptom threshold values 115 that indicate a COVID disease can be established.

The threshold value may be determined, based on the latest medical documentation, such that a value of data obtained below the lowest threshold value is indicative of a low likelihood the patient 102 has contracted a COVID disease. The medical guidelines used to determine predetermined symptom threshold values 115 for all major COVID variants may be updated over time. Therefore, the system for detecting new COVID variants provides a platform that can be updated so the predetermined symptom threshold values 115 reflect the latest understanding of symptoms for major COVID variants. The database comprising predetermined symptom threshold values 115 for all major COVID variants is sent to a machine learning server 116 for further action.

The medical server 113 stores primary instructions for processing data in the database with the patient's biochemical and biophysical data 109 and in the database with predetermined symptom threshold values 115 for all major COVID variants. In an embodiment of the present invention, the primary instructions are executed by the medical server 113 to induce the system for detecting new COVID variants to perform the following steps in accordance with the algorithm: comparing the values of the biochemical and biophysical data 109 to predetermined symptom threshold values 115 for the original COVID-19 virus strain and finding differentials (positive or negative), using the differentials to detect a closely related major COVID variant to the patient's disease (whose symptoms correspond to a majority of the differentials), comparing the values of the biochemical and biophysical data 109 to predetermined symptom threshold values 115 for the closely related major COVID variant and finding differentials (positive or negative).

In another embodiment of the present invention, the primary instructions are executed by the medical server 113 to induce the system to predetermined symptom threshold values 115 for all major COVID variants and find differentials (positive or negative). In another embodiment of the present invention, the closely related major COVID variant is defined such that its symptoms correspond to a minority of differentials detected by comparing the values of the biochemical and biophysical data 109 to predetermined symptom threshold values 115 for the original COVID-19 virus strain.

Set 117 is a set of differentials detected based on the predetermined symptom threshold values 115 for all major COVID variants conforming to medical guidelines that have been obtained by executing the primary instructions. It should be appreciated that this determination can be made by comparing obtained biochemical and biophysical data 109 to at least one known predetermined symptom threshold value 115 indicative of major COVID variant. Differentials can be both positive and negative. The differentials are negative when the values of the data 109 received do not exceed the first predetermined symptom threshold values 115, and positive when the values of the data 109 received exceed the first predetermined symptom threshold values 115. The set of differentials 117 is stored in a database and sent to the analytical server 114 for further analysis, as well as to the machine learning server 116.

The analytical server 114 stores the following databases: a database with differentials 117 that have been determined using the primary instructions, a database with patient's individual physiological data 110, a database with patient's diseases that accompany COVID-19 111, a database with additional patient data 112. The analytical server 114 executes secondary instructions stored on the server, applying them to all data in the databases listed above.

The secondary instructions induce the system to perform the following operations in accordance with the algorithm: grouping the set of differentials 117 into a database of differentials (the database of differentials can be represented as a table of differentials), analyzing all data stored in the databases using the method of combinatorial statistical analysis to detect correlations, analyzing all data stored in the databases using the mathematical method of dense network of curves (method of regression analysis) to detect correlations.

The method of combinatorial statistical analysis and the mathematical method of dense network of curves (method of regression analysis) are both software components that are loaded on the analytical server 114. The resulting set of correlations 118 is stored in a database that is sent to a certification server 119 for further actions and to the machine learning server 116 to create machine learning techniques 120. Also, the database with correlations 118 is stored on a non-transitory computer-readable medium (removable storage drive, a hard disk installed in hard disk drive, flash memories, removable discs, non-removable discs, etc.), a Cloud server, a computer, or any other equivalent device.

The machine learning server 116 stores the following databases: a database with the patient's biochemical and biophysical data 109, a database with predetermined symptom threshold values 115 for all major COVID variants, a database with differentials 117 that have been calculated using the primary instructions, a database with patient's individual physiological parameters 110, a database with patient's diseases that accompany COVID-19 111, a database with additional patient data 112, a database with correlations 118 that have been detected using secondary instructions. The machine learning server 116 applies machine learning techniques 120 to all data stored in the databases listed above.

Additionally, the machine learning server 116 runs analysis (e.g., using combinatorial statistical analysis, cluster analysis, regression analysis) for all data stored in the databases listed above, and the resulting statistics, together with the detected correlations 118, are used in the machine learning techniques 120 to determine possible developments of new COVID variants, post-COVID syndrome, and differences in the courses of diseases caused by major COVID variants depending on the data stored in the databases on the machine learning server 116.

Also, machine learning techniques allow us to set predetermined symptom threshold values 115 for major COVID variants in dependence of the patient's individual physiological parameters 110, and to update and/or adjust predetermined symptom threshold values 115 for major COVID variants. New predetermined symptom threshold values 115 are sent to the medical server 113 to update medical guidelines for major COVID variants. The reports and graphs from machine learning server 116 are stored on the cloud server and certification server 119 for conclusions and suggestions, as well as on a non-transitory computer-readable medium.

Based on the detected correlations that are the mathematical or logical relationships between the values, diagnosis is performed on the certification server 119 having a network connection with the user device 103 (e.g., the smartphone), in which a software application is run. Software can be used to make a medical diagnosis based on the received information to determine the likelihood that the patient 102 has contracted the major COVID variant. The certification server 119 analyses data entries, electronically, to find information to determine if the patient 102 has actually been infected by a major COVID variant or not and to determine whether there is information confirming infection by a major COVID variant. If yes, the certification server 119 generates a diagnosis providing the projected patient's health condition in real-time.

The diagnosis is displayed on the smartphone 103 screen, and the system may determine the likelihood that the patient 102 has contracted the major COVID variant in response to a request by the patient 102 (e.g., via the user device graphical interface). The results provided to the patient 102 could be an indication (positive, negative), the likelihood (1-10, low, medium, high), the disease severity (uninfected, mild, moderate, and severe), etc.

Also, the diagnosis can be provided as a patient's health certificate 121. The patient's health certificate 121 includes a QR code capable of being scanned to display the patient's health certificate 121 on a graphical interface on the user's electronic device 103 (e.g., the smartphone). The patient's health certificate 121 comprises the representation of the patient's biometric sample, which is one or more thumbprint sets, a retina scan, a DNA sample, etc.

The patient's health certificate 121 provides the following information: the patient's viral disease diagnosed, the test result for patient indicating the presence or absence of COVID disease (e.g., antigen test result, molecular test result or antibody test result), the viral risk score, the probability of being infected by a new COVID variant, individual differences between the patient's viral disease diagnosed and the original COVID-19 virus strain, the major COVID variant that is closely related to the patient's disease, the most significant diseases that accompany COVID-19 based on the patient's individual physiological parameters, possible individual traits of the patient's disease course based on the patient's individual physiological parameters, general statistics of the course of the patient's viral disease depending on their sex, age, area of living, etc., the projected post-COVID syndrome that is characteristic for the detected major COVID variant and the patient's individual physiological parameters.

The certification server 119 can be communicatively coupled to an internal API for transmission of patient's health certificates 121 to electronic medical records and human resources records in medical institutions. External APIs can be communicatively coupled to the certification server 119 to query the patient's health certificates 121 associated with the patient 102. For external APIs, the system can output the necessary information based on the type of entity requesting the information. For example, the system can output to a requestor via a graphical representation or report on a smartphone 103 the patient's health certificate 121.

The number of the person's ID 122 for each respective patient 102 can be electronically tied to their corresponding patient's health certificate 121. The person's ID 122 can be used as a unique electronic element or identifier to access subsequent queries for the patient's health certificate 121 of the patient 102. While a preferred embodiment has been set forth above, those skilled in the art will readily appreciate that other embodiments can be realized within the represented diagram of the system.

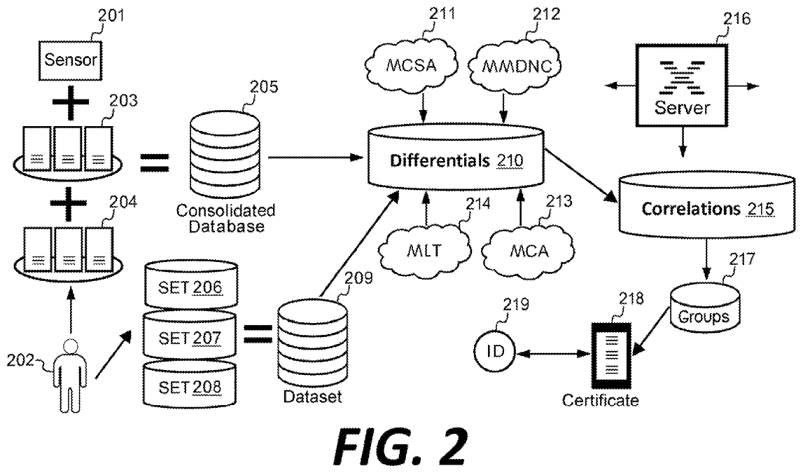

FIG. 2 is a diagram illustrating another example of a system for detecting COVID variants of the present invention. Sensors (including biosensors) 201 collect biochemical and biophysical data from patients 202 for detecting symptoms that indicate the presence of a COVID disease. Also, the biochemical and biophysical data is obtained through laboratory medical tests 203 and laboratory medical examinations 204. Laboratory medical tests include tests for COVID disease that can be antigen tests, molecular tests, or antibody tests. All obtained biochemical and biophysical data is combined into a consolidated database 205.

The patient's individual data is inputted into the system by the patient 202 or by a doctor. The patient's individual data includes patient's individual physiological parameters 206, patient's diseases that accompany COVID-19 207, additional patient data 208. All these sets are stored in dataset 209.

Instructions for calculating differentials have been programmed according to the computer implemented algorithm: comparing the values of the patient's biochemical and biophysical data 205 to predetermined symptom threshold values for the original COVID-19 virus strain (the first SARS-CoV-2 virus strain) to calculate differentials (positive or negative), using the differentials to detect major COVID variant (the second SARS-CoV-2 virus strain) that is closely related to the patient's disease based on a correspondence of its symptoms to the differentials, comparing the values of the patient's biochemical and biophysical data 205 to predetermined symptom threshold values for the closely related major COVID variant (the second SARS-CoV-2 virus strain) to calculate differentials (positive or negative).

The differentials are negative when the values of the patient's biochemical and biophysical data 205 do not exceed the predetermined symptom threshold values, and positive when the values of the patient's biochemical and biophysical data 205 exceed the predetermined symptom threshold values.

In an aspect, the major COVID variant (the second SARS-CoV-2 virus strain) is detected such that its symptoms correspond to a majority of the differential detected by comparing the values of the patient's biochemical and biophysical data 205 to predetermined symptom threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected by comparing the values of the patient's biochemical and biophysical data 205 to predetermined symptom threshold values for the original COVID-19 virus strain (the first SARS-CoV-2 virus strain).

In another embodiment of the present invention, instructions further comprises the step of detecting yet another major COVID variant (the third SARS-CoV-2 virus strain) based on a correspondence of its symptoms to the differentials detected by comparing the values of the patient's biochemical and biophysical data 205 to predetermined symptom threshold values for the closely related major COVID variant (the second SARS-CoV-2 virus strain). Then differentials (positive or negative) are calculated between the values of the patient's biochemical and biophysical data 205 and the predetermined symptom threshold values for yet another major COVID variant (the third SARS-CoV-2 virus strain).

The algorithm may be implemented on the computer (or another smart device, such as a smartphone, tablet, or laptop) or other software (cloud server). When implemented on the smartphone, the algorithm may be a component of the application. When implemented on a computer, the algorithm may be a component of a non-transitory computer-readable medium (removable storage drive, a hard disk installed in a hard disk drive, flash memories, removable discs, non-removable discs, etc.) storing a program of instruction.

A set of all differentials 210 is generated. The set of differentials 210 is analyzed to detect correlations 215. To do this, the set of differentials 210 is complemented by the plurality of the patient's individual data stored in dataset 209 or by the plurality of the patient's biochemical and biophysical data stored in database 205. Then the method of combinatorial statistical analysis 211, the mathematical method of a dense network of curves (method of regression analysis) 212, the methods of cluster analysis 213, the machine learning techniques 214 are used to detect correlations. Correlations are the mathematical or logical relationships between the values within the set 210. The resulting plurality of correlations is stored in a database 215.

Databases that contain a set of patient's biochemical and biophysical data obtained from sensors and through medical tests that include tests for COVID disease (e.g., antigen test, molecular test, antibody test), a set of patient's individual data 209 (patient's individual physiological parameters 206, patient's diseases that accompany COVID-19 207, additional patient data 208), a set of predetermined symptom threshold values for all major COVID variants, a set of differentials, a set of data about possible post-COVID syndromes are generated. The databases are uploaded and stored to the server 216. Then, these databases are analyzed using machine learning techniques 214 that are applied on the data saved on the databases. In another embodiment of the present invention, the databases are uploaded to the cloud server that is shared by multiple computers.

Based on the detected correlations 215 due to the analysis, a patient's viral disease is diagnosed in the event that at least one correlation detected indicates that the patient 202 is likely to have contracted the COVID disease. In another embodiment of the present invention, the determining that the person has the COVID disease is also based on a confirmation of its symptoms with the values of the patient's biochemical and biophysical data 205 collected from patients 202.

In another embodiment of the present invention, the detected correlations stored in a database 215 are further analyzed using cluster analysis 213 to define the same or similar correlations and combining these correlations into a group 217. The cluster analysis uses the differences in the correlations detected to define multiple groups of correlations 217 that are same or similar. Based on the multiple groups of correlations 217 detected, a patient's viral disease is diagnosed in the event that at least one detected group of correlations indicates that the patient 202 is likely to have contracted the COVID disease.

In response to a determination that the patient 202 has or has not contracted the disease, a diagnosis indicating the presence or absence of a disease is generated. The diagnosis can be a test result indicating the presence or absence of COVID disease, e.g., antigen test result, molecular test result, or antibody test result.

The system generates diagnosis in the form of a patient's health certificate 218, which comprises the patient's viral disease as well as all information on the diagnosis, including the test result for COVID disease (e.g., antigen test result, molecular test result, or antibody test result). The patient's health certificate 218 includes a QR code capable of being scanned on a user interface and can be tied to the person's ID 219 and used for various digital identifications of the patient 202. Or the number of the person's ID 219 for each respective patient 202 can be electronically tied to their corresponding patient's health certificate 218.

In an aspect, the patient's health certificate 218 is storied to a database on a computer for further outputting or displaying. In another aspect, the patient's health certificate 218 is transmitted to a mobile electronic device using end-to-end encryption. In yet another aspect, the patient's health certificate 218 is loaded to a Cloud server that is shared by multiple computers.

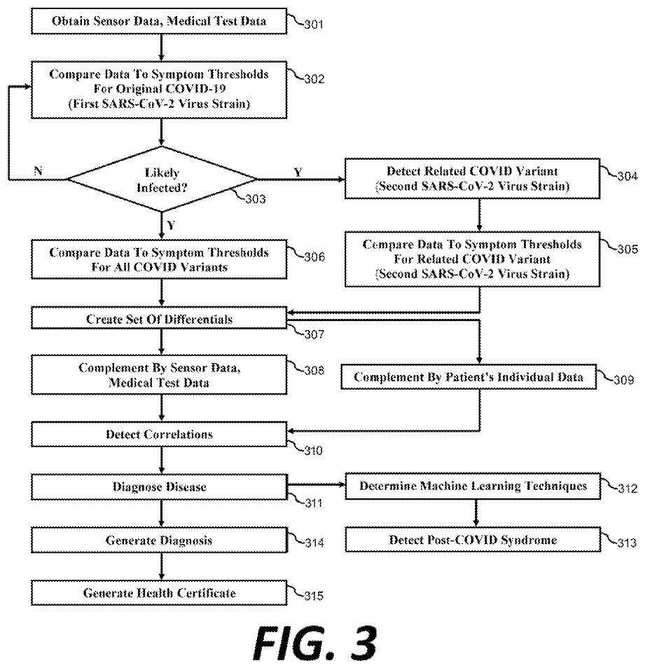

FIG. 3 is a flowchart illustrating the steps of the algorithm for detecting COVID variants according to a first embodiment of the present invention. The patient's biochemical and biophysical data is obtained from a variety of sensors in step 301. In another aspect, the medical institution runs laboratory medical tests for COVID disease, laboratory medical examinations for COVID disease and so obtains biochemical and biophysical data in step 301.

The sensors may include a smartphone, a pulse oximeter, a body temperature thermometer, etc. The sensors also may include biosensors. The data is transferred by the sensor using a secure encoded channel. The process may be performed by the server or central processing unit in conjunction with the user device (e.g., running a software program provided by the server or central processing unit). The sensor data may provide direct evidence the user is experiencing one of the symptoms.

Then, the values of the patient's biochemical and biophysical data obtained is compared to predetermined symptom threshold values for the original COVID-19 virus strain (the first SARS-CoV-2 virus strain) in step 302, e.g., the values of the patient's biochemical and biophysical data obtained through laboratory medical tests are compared to positive and negative predetermined IGM and IGG antibody values indices used as references for determination viral diseases, particularly, COVID disease. Based on this comparison, the probability of the patient having this viral disease is assessed, and it is concluded whether the patient is infected with COVID disease or not in step 303. Therefore, the patient's viral disease is identified.

However, the process does not stop here and returns to steps 301, in which more patient's biochemical and biophysical data is obtained from sensors and through laboratory medical tests, laboratory medical examinations. This updated patient data is again compared with predetermined symptom threshold values in step 302. Additionally, the set of the patient's individual physiological parameters, such as age, sex, gender, blood type, blood pressure, blood sugar, immunity, vaccination history, etc., is generated. This set may also include diseases that accompany COVID-19, e.g., tuberculosis, diabetes, severe immunosuppression, lymphoma, oncological diseases, ulcers, cardiovascular pathologies, nervous diseases, etc.

Thus, the values of the patient's biochemical and biophysical data obtained from sensors and through laboratory medical tests, laboratory medical examinations are compared to predetermined symptom threshold values for the original COVID-19 virus strain (the first SARS-CoV-2 virus strain) in step 302. Differences are calculated, and resulting differentials, both positive and negative, are recorded. The differentials are negative when values of the patient's biochemical and biophysical data obtained do not exceed the predetermined symptom threshold values, and positive when values of the patient's biochemical and biophysical data obtained exceed the predetermined symptom threshold values.

If a resulting differential is positive, it means the value of the patient's biochemical and biophysical data exceeds the predetermined symptom threshold value for the original COVID-19 virus strain (the first SARS-CoV-2 virus strain), and the diagnostic follows the algorithm disclosed herein. If a resulting differential is negative, it means the patient's biochemical and biophysical data is below the threshold, and the process returns to step 301, in which additional data is obtained from using sensors and/or using new laboratory medical tests (and/or laboratory medical examinations), which are run by the medical institution.

In an embodiment of the present invention, in step 304, the differentials (positive or negative) obtained in step 302 by comparing the values of the patient's biochemical and biophysical data to predetermined symptom threshold values for the original COVID-19 virus strain (the first SARS-CoV-2 virus strain) are used to identify a major COVID variant (the second SARS-CoV-2 virus strain) that is closely related to the patient's viral disease diagnosed in step 303. The closely related major COVID variant (the second SARS-CoV-2 virus strain) is detected based on a correspondence of its symptoms to the differentials calculated in step 302.

In an aspect, the closely related major COVID variant (the second SARS-CoV-2 virus strain) is detected such that its symptoms correspond to a majority of the differential. In another aspect, the closely related major COVID variant (the second SARS-CoV-2 virus strain) is detected such that its symptoms correspond to a minority of the differential. Then, the values of the patient's biochemical and biophysical data is compared to predetermined symptom threshold values for the closely related major COVID variant (the second SARS-CoV-2 virus strain) in order to calculate differentials (positive or negative) in step 305.

In another embodiment of the present invention, when obtaining differentials in step 302 by comparing the values of the patient's biochemical and biophysical data to predetermined symptom threshold values for the original COVID-19 virus strain (the first SARS-CoV-2 virus strain), the patient data obtained is additionally compared to predetermined symptom threshold values for all major COVID variants in step 306 to calculate differentials (positive or negative) for all major COVID variants.

All differentials are grouped in step 307 into the set of differentials (for example, differentials can be combined into the table of differentials for further analysis). In an embodiment of the present invention, in step 308, this set of differentials is complemented by the set of the patient's biochemical and biophysical data obtained. In another embodiment of the present invention, in step 309, this set of differentials is complemented by the set of patient's individual physiological parameters, including diseases that accompany COVID-19.

The complemented set of differentials is then analyzed in step 310 using statistical methods (e.g., the method of combinatorial statistical analysis, the methods of cluster analysis), mathematical methods (e.g., the mathematical method of dense network of curves) to detect correlations within the set. The correlations indicative of relationships between within the values of the complemented set of differentials.

Based on the detected correlations in step 310 that are the mathematical or logical relationships between the values, a viral disease is diagnosed in step 311, wherein the following processes are involved: diagnosing the patient's viral disease and getting test result indicating the presence or absence of COVID disease (e.g., antigen test result, molecular test result or antibody test result), detecting a major COVID variant (the second SARS-CoV-2 virus strain) that is closely related to the patient's viral disease diagnosed (the process returns to steps 302 and 304), keeping statistics of the viral disease course depending on the set of differentials (the process returns to steps 303 and 307), predicting the disease course based on the set of differentials and patient's individual physiological parameters (the process returns to steps 307 and 309), determining individual traits of the course of the patient's diseases that accompany COVID-19 (the process returns to steps 303 and 309), predicting the post-COVID syndrome and its idiosyncrasies (the process returns to steps 309 and 311).

Based on the resulting diagnosis, machine learning techniques are determined in step 312 for the set of differentials and the set of patient's individual physiological parameters, including diseases that accompany COVID-19, in order to determine individual traits of the disease course (the process can be return to steps 303 and 311). In another embodiment of the present invention, machine learning techniques are used to update and/or adjust predetermined symptom threshold values for all major COVID variants (the process returns to steps 302 and 306). In another embodiment of the present invention, machine learning techniques are used to adjust predetermined symptom threshold values for all major COVID variants, considering the patient's individual physiological parameters that include diseases that accompany COVID-19 (the process returns to steps 306 and 309).

Using machine learning techniques the post-COVID syndrome that can be expected for the identified major COVID variant, taking into account the patient's individual physiological parameters, including diseases that accompany COVID-19, is predicted in step 313. For example, the post-COVID syndrome for the Delta variant often involved increased fatigue, long-term nasopharyngeal inflammation, voice changes, impaired memory, cognitive failures (slower reaction, inability to operate properly, etc.), impaired hearing, intestinal disorders, lung and heart lesions, increased susceptibility to other infections.

A system generates a diagnosis in step 314. The diagnosis is displayed on the smartphone screen in real-time, showing the risk of the patient being infected by a major COVID variant. In step 315, the diagnosis is represented as a patient's health certificate, in which the patient's viral disease is given. The patient's health certificate comprises the representation of the biometric sample of the patient. The biometric sample is one or more of a thumbprint set recorded from the patient, a retina scan recorded from the patient, and a DNA sample obtained from the patient and analyzed, etc.

The patient's health certificate provides the patient's detected viral disease, the test result for the patient that indicates the presence or absence of COVID disease (e.g., antigen test result, molecular test result or antibody test result), the viral risk score, the probability of the patient being infected by a COVID variant, individual differences between the patient's viral disease diagnosed and the original COVID-19 virus strain (the first SARS-CoV-2 virus strain), the major COVID variant (the second SARS-CoV-2 virus strain) that is closely related to the patient's disease, possible individual traits of the patient's disease course based on the patient's individual physiological parameters, general statistics of the course of the patient's disease depending on their sex, age, area of living, etc., the projected post-COVID syndrome.

The certification server can be communicatively coupled to an internal API for transmission of health certificates to electronic medical records and human resources records in medical institutions. External APIs can be communicatively coupled to the certification server to query the health certificates associated with the patient. For external APIs, the system can output the necessary information based on the type of entity requesting the information, for example, to access a specific venue, which is any public place with a large number of people, where permission to enter is required and where the chance of a spread of a viral infection is greater.

The generated patient's health certificate can be tied to the person's ID and used for various digital identifications of the patient. The person's ID number for each respective patient can be electronically tied to their corresponding health certificate, and then the person ID can be used as a unique electronic element or identifier to access with subsequent queries for a health certificate of the patient. The system can output the necessary information based on the type of entity requesting the information, and can output to a requestor an indication the patient has or does not have a viral disease COVID variant. For this, the patient's health certificate includes a code (e.g., a QR code) capable of being scanned to display the health certificate on user interfaces or an electronic device.

In some embodiments of the present invention, step 309 has an additional step, in which, based on the analysis of the set of differentials and the set of the patient's individual physiological parameters, a major COVID variant (the second SARS-CoV-2 virus strain) is identified, which is closely related to the patient's viral disease that has been diagnosed in step 303. Then, the process returns to step 305, in which the biochemical and biophysical data is compared with the predetermined symptom threshold values for the related major COVID variant (the second SARS-CoV-2 virus strain) to calculate differentials. Then, a new set of differentials is generated and analyzed using statistical methods, mathematical methods, machine learning techniques to detect correlations that are the mathematical or logical relationships between the values within the set. Based on the correlations, the viral disease is diagnosed again, but with higher precision.

In some embodiments of the present invention, according to the algorithm, databases with patient's biochemical and biophysical data, patient's individual physiological parameters, diseases that accompany COVID-19, additional patient data, predetermined symptom threshold values for the original COVID-19 virus (the first SARS-CoV-2 virus strain), and predetermined symptom threshold values for at least one major COVID variant (the second SARS-CoV-2 virus strain) are generated. Then, these databases are analyzed using statistical methods, mathematical methods, machine learning techniques that are applied on all data saved on the databases. Based on the results of the analysis and detected correlations, the viral disease is diagnosed again, but with higher accuracy.

In some embodiments of the present invention, the tests for COVID disease are conducted and results are awaited. According to the algorithm, the system can determine the IGG antibody index for the patient in step 301, the system can determine any prior conditions associated with the patient in step 303, then again the system can determine an IGM antibody index for the patient in step 301, and the system determines the patient's individual physiological parameters in step 309. The data can include manual testing and/or automated testing results, both in real-time and previously performed tests.

Steps 302 and 306 can incorporate medical guidelines associated with predetermined symptom threshold values for all major COVID variants to determine whether the IGG index or the IGM index, respectively, are at levels below or above the predetermined symptom threshold value. If tested positive, differentials are automatically determined for all major COVID variants. Also, again the same procedure is followed for the patient's individual physiological parameters. Whenever a patient tests positive, the system will list the data of differentials and data of the patient's individual physiological parameters.

The IGG index, IGM index, differentials, and the patient's individual physiological parameters can be used to generate a risk score or level. The risk score or level can be updated in a real-time or substantially real-time manner as additional test data is obtained and/or as medical guidelines are updated. The information is saved in step 312 on the database and that data is analyzed using machine learning techniques. The reports and graphs from machine learning computers are stored on a cloud server for conclusions and suggestions. While a preferred embodiment has been set forth above, those skilled in the art will readily appreciate that other embodiments can be realized within the flowchart of the algorithm.

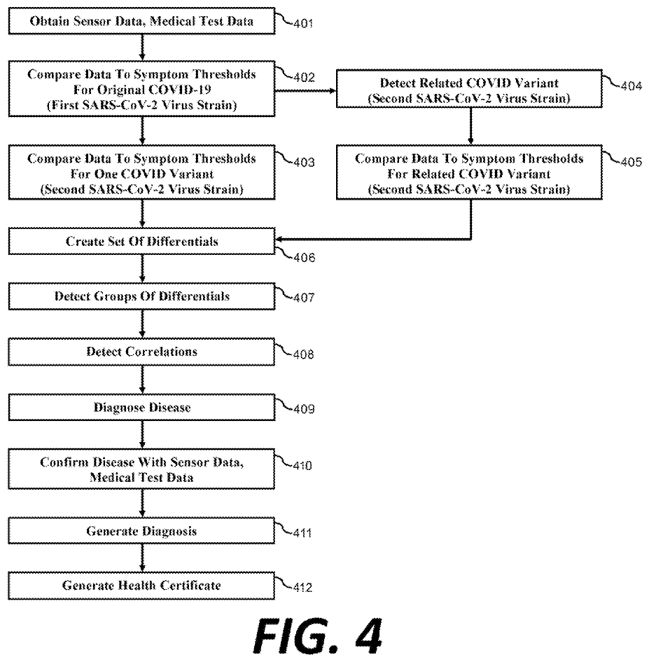

FIG. 4 is a flowchart illustrating the steps of the algorithm for detecting COVID variants according to a second embodiment of the present invention. The system of the present invention for detecting new COVID variants includes a group of servers (a central server, a medical server, an analytical server, a machine learning server, a certification server), on which operations are performed according to the algorithm, comprising the following steps.

At least one patient's biochemical and biophysical data is obtained in step 401 from the sensors and through the medical tests that can be tests for COVID disease (e.g., antigen test, molecular test, antibody test). The values of the patient's biochemical and biophysical data are compared to predetermined symptom threshold values for the original COVID-19 virus strain (the first SARS-CoV-2 virus strain) in step 402 to calculate differentials (positive or negative). The values of the patient's biochemical and biophysical data are compared to predetermined symptom threshold values for at least one major COVID variant (the second SARS-CoV-2 virus strain) in step 403 to calculate differentials (positive or negative).

In another embodiment of the present invention, in step 404, the major COVID variant (the second SARS-CoV-2 virus strain) that is closely related to the patient's disease is detected based on a correspondence of its symptoms to the differentials detected in step 402 by comparing the values of the patient's biochemical and biophysical data to predetermined symptom threshold values for the original COVID-19 virus strain (the first SARS-CoV-2 virus strain). The values of the patient's biochemical and biophysical data are compared to predetermined symptom threshold values for the closely related major COVID variant (the second SARS-CoV-2 virus strain) in step 405 to calculate differentials (positive or negative).

In an aspect, the closely related major COVID variant (the second SARS-CoV-2 virus strain) is detected such that its symptoms correspond to a majority of the differential detected in step 402 by comparing the values of the patient's biochemical and biophysical data to predetermined symptom threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected in step 402 by comparing the values of the patient's biochemical and biophysical data to predetermined symptom threshold values for the original COVID-19 virus strain (the first SARS-CoV-2 virus strain).

The set of all differentials is created in step 406. In step 407, the differentials are combined within the set of differentials into multiple groups based on the differences in the differentials. The differentials not included in the groups are not taken into account in the further analysis of the set of differentials. Based on the multiple groups of differentials, the set of differentials are analyzed in step 408 to detect correlations indicative of relationships between the groups of the differentials. Based on the detected correlations, a patient's viral disease is diagnosed in step 409 in the event that at least one detected correlation indicates that the patient is likely to have contracted the COVID disease.

The determining in step 410 that the patient has the viral disease is based on a confirmation of its symptoms with the values of the patient's biochemical and biophysical data obtained from the sensors and through the medical tests in step 401. In response to a determination that the patient has or has not contracted the disease, a diagnosis indicating the presence or absence of a disease is generated in step 411. The diagnosis can be a test result indicating the presence or absence of COVID disease in a patient.

Based on the received diagnosis, in step 412, the system generates a patient's health certificate, which includes the patient's disease, the test result for the patient that indicates the presence or absence of COVID disease (e.g., antigen test result, molecular test result, or antibody test result), the viral risk score, the difference between the disease and the original COVID-19 virus, the probability of the patient being infected by a new COVID variant, the major COVID variant that is closely related to the disease, patient's diseases that accompany COVID-19, the disease statistics by criterion (e.g., sex, age, region), the projected post-COVID syndrome for the patient. In an aspect, the patient's health certificate is storied to a database on a computer for further outputting or displaying. In another aspect, the patient's health certificate is transmitted to a mobile electronic device using end-to-end encryption. In yet another aspect, the patient's health certificate is loaded to a Cloud server shared by multiple computers.

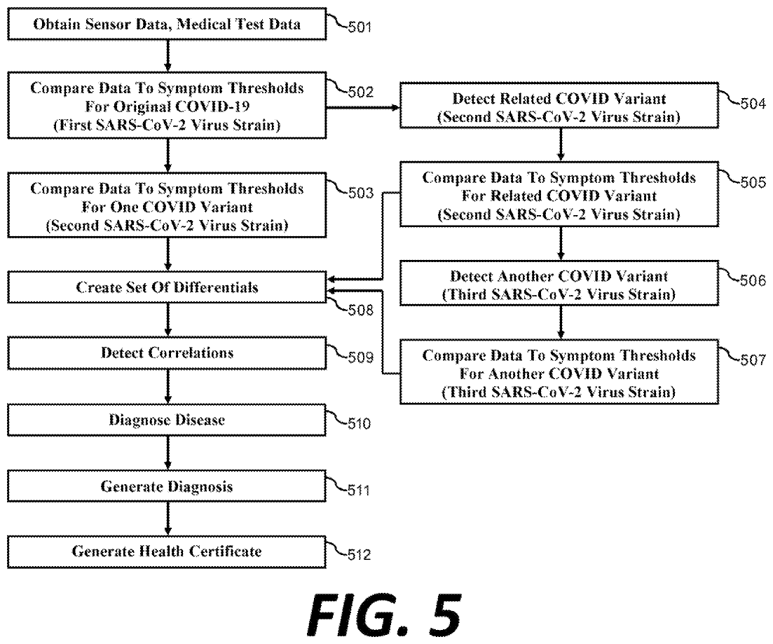

FIG. 5 is a flowchart illustrating the steps of the algorithm for detecting COVID variants according to a third embodiment of the invention. At least one patient's biochemical and biophysical data is obtained in step 501 from the sensors and through the medical tests that can be tests for COVID disease (e.g., antigen test, molecular test, antibody test). The values of the patient's biochemical and biophysical data are compared to predetermined symptom threshold values for the original COVID-19 virus strain (the first SARS-CoV-2 virus strain) in step 502 to calculate differentials (positive or negative). The values of the patient's biochemical and biophysical data are compared to predetermined symptom threshold values for at least one major COVID variant (the second SARS-CoV-2 virus strain) in step 503 to calculate differentials (positive or negative).

In step 504, the major COVID variant (the second SARS-CoV-2 virus strain) that is closely related to the patient's disease is detected based on a correspondence of its symptoms to the differentials detected in step 502 by comparing the values of the patient's biochemical and biophysical data to predetermined symptom threshold values for the original COVID-19 virus strain (the first SARS-CoV-2 virus strain). The values of the patient's biochemical and biophysical data are compared to predetermined symptom threshold values for the closely related major COVID variant (the second SARS-CoV-2 virus strain) in step 505 to calculate differentials (positive or negative).

In an aspect, the closely related major COVID variant (the second SARS-CoV-2 virus strain) is detected such that its symptoms correspond to a majority of the differential detected in step 502 by comparing the values of the patient's biochemical and biophysical data to predetermined symptom threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected in step 502 by comparing the values of the patient's biochemical and biophysical data to predetermined symptom threshold values for the original COVID-19 virus strain (the first SARS-CoV-2 virus strain).

The algorithm further comprises the step of using the differentials for the closely related COVID variant for detecting yet another major COVID variant (the third SARS-CoV-2 virus strain). In step 506, the yet another major COVID variant (the third SARS-CoV-2 virus strain) is detected based on a correspondence of its symptoms to the differentials detected in step 505 by comparing the values of the patient's biochemical and biophysical data to predetermined symptom threshold values for the closely related major COVID variant (the second SARS-CoV-2 virus strain). The values of the patient's biochemical and biophysical data are compared to predetermined symptom threshold values for yet another major COVID variant (the third SARS-CoV-2 virus strain) in step 507 to calculate differentials (positive or negative).

In an aspect, the yet another major COVID variant (the third SARS-CoV-2 virus strain) is detected such that its symptoms correspond to a majority of the differential detected in step 505 by comparing the values of the patient's biochemical and biophysical data to predetermined symptom threshold values for the closely related major COVID variant (the second SARS-CoV-2 virus strain). In another aspect, the yet another major COVID variant (the third SARS-CoV-2 virus strain) is detected such that its symptoms correspond to a minority of the differentials detected in step 505 by comparing the values of the patient's biochemical and biophysical data to predetermined symptom threshold values for the closely related major COVID variant (the second SARS-CoV-2 virus strain).

The set of all differentials is created in step 508. The set of differentials are analyzed in step 509 to detect correlations that are the mathematical or logical relationships between the values within set of differentials. Based on the detected correlations, a patient's viral disease is diagnosed in step 510 in the event that at least one detected correlation indicates that the patient is likely to have contracted the COVID disease. In response to a determination that the patient has or has not contracted the disease, a diagnosis indicating the presence or absence of a disease is generated in step 511. Based on the received diagnosis, the system generates a patient's health certificate in step 512.

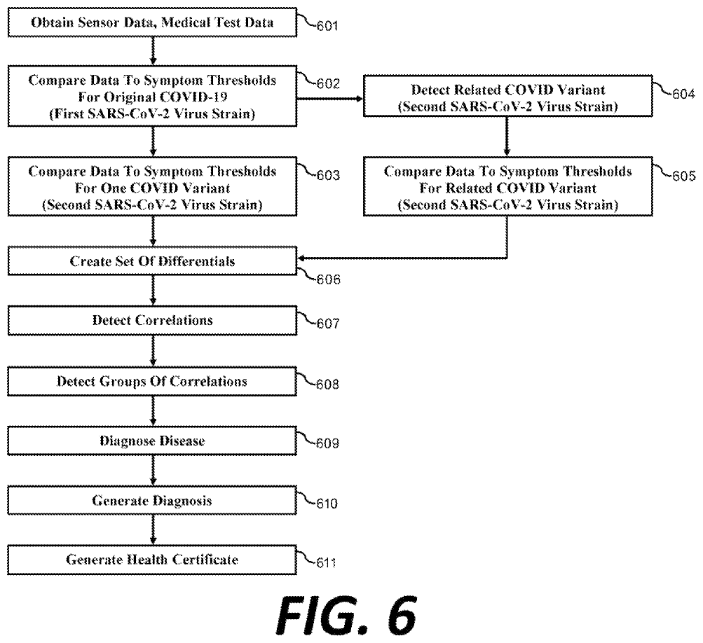

FIG. 6 is a flowchart illustrating the steps of the algorithm for detecting COVID variants according to a fourth embodiment of the invention. At least one patient's biochemical and biophysical data is obtained in step 601 from the sensors and through the medical tests that can be tests for COVID disease (e.g., antigen test, molecular test, antibody test). The values of the patient's biochemical and biophysical data are compared to predetermined symptom threshold values for the original COVID-19 virus strain (the first SARS-CoV-2 virus strain) in step 602 to calculate differentials (positive or negative). The values of the patient's biochemical and biophysical data are compared to predetermined symptom threshold values for at least one major COVID variant (the second SARS-CoV-2 virus strain) in step 603 to calculate differentials (positive or negative).

In step 604, the major COVID variant (the second SARS-CoV-2 virus strain) that is closely related to the patient's disease is detected based on a correspondence of its symptoms to the differentials detected in step 602 by comparing the values of the patient's biochemical and biophysical data to predetermined symptom threshold values for the original COVID-19 virus strain (the first SARS-CoV-2 virus strain). The values of the patient's biochemical and biophysical data are compared to predetermined symptom threshold values for the closely related major COVID variant (the second SARS-CoV-2 virus strain) in step 605 to calculate differentials (positive or negative).

In an aspect, the closely related major COVID variant (the second SARS-CoV-2 virus strain) is detected such that its symptoms correspond to a majority of the differential detected in step 602 by comparing the values of the patient's biochemical and biophysical data to predetermined symptom threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected in step 602 by comparing the values of the patient's biochemical and biophysical data to predetermined symptom threshold values for the original COVID-19 virus strain (the first SARS-CoV-2 virus strain).

The set of all differentials is created in step 606. The set of differentials are analyzed in step 607 to detect correlations that are the mathematical or logical relationships between the values within set of differentials. The algorithm further comprises the step 608 of analyzing the detected correlations to define the same or similar correlations and combining these correlations into a group. Based on the detected multiple groups of correlations, a patient's viral disease is diagnosed in step 609 in the event that at least one detected group of correlations indicates that the patient is likely to have contracted the COVID disease. In response to a determination that the patient has or has not contracted the disease, a diagnosis indicating the presence or absence of a disease is generated in step 610. Based on the received diagnosis, the system generates a patient's health certificate in step 611.

While various embodiments of the algorithm have been described in FIGS. 3-6, in general case, the algorithm of the present invention comprises the steps: receiving symptom data values from patient, calculating first differentials (positive or negative) for the first COVID virus strain by comparing the values to first predetermined symptom threshold values for the first COVID virus strain, using the first differentials to detect a second COVID virus strain with a mutated virus genome code based on a correspondence of their symptoms to the first differentials, calculating second differentials (positive or negative) for the second COVID virus strain by comparing the values to second predetermined symptom threshold values for the second COVID virus strain, creating a set of the first and second differentials, detecting correlations within the set, determining that the patient has the first or second COVID virus strain when at least one detected correlation indicates the patient has contracted the first or second COVID virus strain, outputting a result indicating a presence or absence of COVID disease in a patient.

The algorithm may be implemented by a non-transitory computer-readable medium, a central processing unit, server, cloud server, computer, smartphone, or another smart device, such as a tablet or laptop. The algorithm of the present invention is different from other traditional methods in modern medical practice regarding the determining of COVID disease.

According to conventional medical practice, viral diseases are identified using antibody tests, such as tests for high amounts of IGG or low amounts of IGM. Such tests are also one of the main conventional methods for detecting and diagnosing COVID disease, just like real-time reverse-transcriptase-polymerase chain reaction (rRT-PCR) tests. Other methods are used, in which patient's biochemical and biophysical data that has been obtained from a variety of sensors are identified and then compared with predetermined symptom threshold values to detect a viral disease.

These methods have the following drawbacks. They don't provide enough precision when diagnosing a viral disease, and they don't take into account the severity of the disease, or idiosyncrasies of its course in relation to individual physiological parameters of the patient (e.g., their natural immunity). However, in many cases, the course of the COVID disease is quite unique for each person, depending on their physiological parameters. Besides, newer mutated COVID variants have changed virus genome codes and, therefore, don't affect the human organism in the same way as the original COVID-19 (SARS-CoV-2) virus strain.

Also, for instance, according to conventional medical practice, differentials are calculated as differences between the data of an ill person and the data of a healthy person. In the present invention, however, it is proposed to calculate differentials (differences) between the values of the patient data obtained from plurality of sensors, through laboratory medical tests and predetermined symptom threshold values for a COVID variant, wherein differentials are calculated for all known major COVID variants: Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc.

These differences may be not only positive, i.e., when the patient's biochemical and biophysical data exceeds predetermined symptom threshold values, but also negative, i.e., when the patient data obtained is lower than the threshold values. In the latter case, according to the conventional medical practice, a viral disease cannot be diagnosed, and therefore, the COVID disease is not identified. In the present invention, it is proposed that all differentials, i.e., both positive and negative (the differentials are negative when the values of the date obtained do not exceed the first predetermined symptom threshold values, and positive when the values of the date obtained exceed the first predetermined symptom threshold values), are calculated and then combined into a set of differentials for further analysis and detecting correlations between the values within the set of differentials that indicate the patient has contracted the COVID disease.

This set of the values of differentials is analyzed and complemented, in an embodiment of the present invention, by the plurality of the values of the biochemical and biophysical data obtained, or, in another embodiment of the present invention, by the plurality of the values of patient's individual physiological parameters, such as their age, sex, gender, blood type, blood pressure, blood sugar, immunity, lung or heart diseases, etc. to detect correlations within the values of the set.

The detected correlations can be used to: diagnose the patient's disease (e.g., get test result that indicate the presence or absence of a COVID disease in a patient), detect major COVID variant that can be closely related to the viral disease diagnosed, keep statistics of the viral disease course depending on its differentials, predict the patient's disease course based on their differentials and individual physiological parameters, create machine learning techniques for the viral disease course idiosyncrasies in relation to the set of differentials and patient's individual parameters, determine idiosyncrasies of the course of the patient's diseases that accompany COVID-19, detect the post-COVID syndrome based on the patient's individual physiological parameters.

The present invention includes at least two main advantages of the prior art that need further improvement. Firstly, it would allow to generate computerized databases with differentials for all major COVID variants in relation to the patient's individual physiological parameters. These databases would be supported by medical institutions and used to detect and thoroughly analyze any new mutated COVID variants. Secondly, it would allow the diagnosis of viral diseases in a more standardized and efficient way by using specific data combining differentials with patient's individual physiological parameters and patient's diseases that accompany COVID-19. The present invention has other differences from prior art documents.

For example, U.S. Pat. No. 10,902,955 B1, entitled “Detecting COVID-19 Using Surrogates” (incorporated herein by reference), discloses “a method for determining whether a user is likely to have contracted a disease, the method comprising: receiving sensor data from a user device; receiving and storing locations of the user device; for each of a plurality of symptoms of the disease, calculating a symptom metric indicative of the likelihood that the user is experiencing the symptom, wherein, at least some of the plurality of symptom metrics are calculated by comparing the sensor data to a predetermined baseline and comparing a difference between the sensor data and the predetermined baseline to at least one predetermined symptom threshold, and at least one of the symptoms is anosmia or ageusia and the symptom metric indicative of the likelihood that the user is experiencing anosmia or ageusia is determined by analyzing sound recorded by a microphone using a speech detection algorithm to identify phrases in the recorded sound indicative of anosmia or ageusia; weighing each of the symptom metrics to form a composite metric indicative of the likelihood that the user is likely to have contracted the disease; determining whether the user is likely to have contracted the disease by comparing the composite metrics to one or more predetermined composite thresholds; and in response to a determination that the user is likely to have contracted the disease, identifying additional devices in the location of the user device and outputting information to the users of the additional devices.”

Therefore, some of the pluralities of symptom metrics are calculated by comparing the sensor data to a predetermined baseline, and comparing a difference between the sensor data and the predetermined baseline to at least one predetermined symptom threshold, wherein the baseline reflects symptom values characteristic for a healthy person. Therefore, differentials mentioned in the cited prior art document reflect differences between actual values for an infected person with base values for a healthy person. However, other differentials are calculated according to the present invention, particularly, differentials that are differences between the values of biochemical and biophysical data obtained and predetermined symptom threshold values for major COVID variants.

In addition, the cited document clearly shows that differentials calculated can be only positive, as differences are taken into account only if the values are above the baseline. According to one aspect of the present invention, differentials can also be negative, and negative differentials are analyzed just as any other differential, in order to detect correlations.

In addition, the cited document discloses mandatory steps of generating combined symptom metrics, calculating complex values based on these metrics, and determining infection probability. The patent discloses that “the difference between the user health metrics (body temperature, heart rate, or heartrate variability) and the (generalized or personalized) baseline is then converted into a single unitary metric indicative of the presence of (and, preferably, the probability or severity of) a fever. In some embodiments, the difference between the user health metric and the baseline may be compared to a threshold determined by the COVID-19 triage system to be indicative of the presence of a fever or multiple thresholds determined by the COVID-19 triage system to be indicative of the probability or severity of a fever”.

However, the present invention uses a completely different method for diagnosing COVID disease, comprising the steps: comparing the values of the patient data obtained to predetermined symptom threshold values for the original COVID-19 virus strain to calculate differentials (positive or negative), using the differentials to detect a closely related major COVID variant to the patient's disease based on a correspondence of their symptoms to the differentials, comparing the values of the patient data obtained to predetermined symptom threshold values for the closely related major COVID variant to calculate differentials (positive or negative), generating a set of all differentials, analyzing the set to detect correlations between the values within the set, diagnosing the patient's viral disease in the event that at least one detected correlation indicates the patient has contracted the COVID disease. This is a completely different approach to detecting a COVID disease.

U.S. Pat. No. 11,024,339 B2, entitled “System and Method for Testing for COVID-19” (incorporated herein by reference), discloses “a method for using at least a mobile computing device to detect coronavirus in a user, comprising the steps of: receiving by the mobile computing device pulse and oxygen saturation level data from a pulse oximeter attached to the user periodically over a period of time, resulting in a plurality of sets of the pulse oximeter data; receiving movement data during the period of time, the movement data being indicative of at least movement of the user with respect to at least one fixed location; identifying at least one of the plurality of sets of pulse oximeter data as being accurate when the movement data indicates that the movement of the user with respect to the fixed location is equal to or less than a pre-determined amount; storing at least the accurate set of pulse oximeter data on the user and comparing it to previously stored pulse oximeter data from the user to determine a differential; comparing the differential to at least one known value; and using at least the comparison to determine whether the user is suffering from coronavirus.”

In that patent, the differences between actual data and precise data are calculated. Some data is identified as precise, and then the differences are calculated. This is basically the same as calculating differentials in the previous prior art document referenced above. For example, this patent discloses that “an increased heartrate is an indication of fever. Therefore, the COVID-19 system may detect a fever by detecting the user's heart rate and comparing the detected heart rate to the baseline heart rate for the user.”

But according to the present invention, a completely different differential is calculated and used. The differential is differences between the values of biochemical and biophysical data obtained and predetermined symptom threshold values for major COVID variants. These two approaches are completely different in methodology since the differential in the present invention is calculated in relation to predetermined symptom threshold values for major COVID variants (i.e., to predetermined symptom threshold values for a patient infected by any one of the major COVID variants), rather than in relation to predetermined symptom threshold values for a healthy person.

Also, differentials in the present invention can be both positive and negative (when the patient data obtained is below predetermined symptom threshold values). Negative differentials are analyzed together with other differentials, and then the resulting set of differentials, the set of the values of the biochemical and biophysical data obtained, the set of patient's individual physiological parameters are analyzed in order to detect new COVID variants. Also the present invention does not comprise the steps of “receiving movement data during a period of time of the user with respect to fixed location” or “identifying plurality of sets of data as being accurate when movement data indicates that movement of the user with respect to fixed location is equal to or less than a pre-determined amount.”

The proposed method of the present invention for detecting new COVID variants comprises different steps, such as: comparing the values of the patient data obtained to predetermined symptom threshold values for the original COVID-19 virus strain to calculate differentials (positive or negative), using the differentials to detect a closely related major COVID variant to the patient's disease based on a correspondence of their symptoms to the differentials, comparing the values of the patient data obtained to predetermined symptom threshold values for the closely related major COVID variant to calculate differentials (positive or negative), generating a set of all differentials, analyzing the set to detect correlations between the values within the set, diagnosing the patient's viral disease in the event that at least one detected correlation indicates the patient has contracted the COVID disease.

Thus, the methods and approaches of the present invention are different from conventional documents. Steps that involve comparing the values of patient data obtained with predetermined symptom threshold values for the original COVID-19 virus strain and new mutated major COVID variants that have changed genome codes, rather than with a “healthy person” standard, are added. This novel method would allow significantly increased precision of disease diagnosis, since mutated COVID variants would share parts of a mutated virus genome code of a previous variant and would repeat some of the same symptoms.

By systematizing and analyzing differentials, it would be possible to separate the symptoms, affected areas, and disease course of the original variant from new ones that may affect a person differently. In addition, this would allow calculation of individual morbidity risks for a person taking into account their individual physiological parameters and diseases that accompany COVID-19, and also to determine a possible disease course and individual traits of post-COVID syndrome, e.g., if a person has a certain amount of antibodies from a previous COVID variant. This would also allow to create new machine learning techniques, in case a person is infected by a new COVID variant.

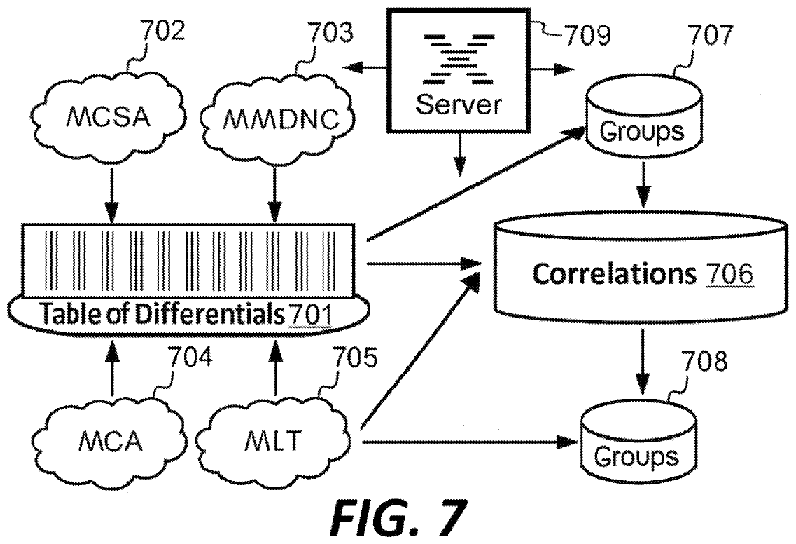

FIG. 7 is a diagram illustrating the analysis of medical data of the present invention. The set of differentials is grouped into the table of differentials 701 and stored on a server 709 or a Cloud server. The table 701 comprises all differentials obtained for major COVID variants, which are then processed using the method of combinatorial statistical analysis 702, the mathematical method of dense network of curves 703, the methods of cluster analysis 704, the machine learning techniques 705 to detect correlation 706.

In another aspect, the differentials within the table of differentials 701 are combined into multiple groups of differentials 707 based on the differences in the differentials. The differentials that were not included in the groups 707 are not taken into account in the further analysis of the set of differentials. Based on the multiple groups of differentials 707 detected, the set of differentials is analyzed using these methods to detect correlations 706 indicative of relationships between the groups of the differentials.

A set of the all detected correlation 706 are created. The correlations within the set are further analyzed by using the methods of cluster analysis 704 to define the same or similar correlations and combining these correlations into a group 708. The correlations that were not included in the groups are not taken into account in the further determination of COVID disease in a patient. Thus the multiple groups of correlations 708 having same or similar correlations are created.

All the data is stored in the databases on the server 709. Then, these databases are analyzed by using machine learning techniques 705 that are applied on the data saved on the databases. In another embodiment of the present invention, the databases are uploaded to the cloud server that is shared by multiple computers.

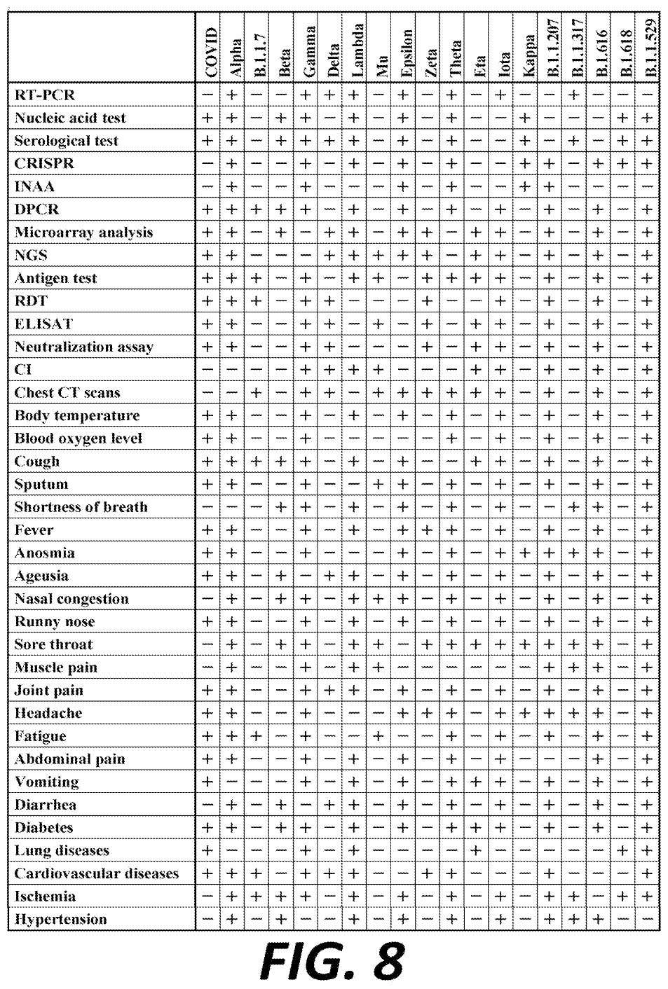

FIG. 8 illustrates the table of differentials 701 of FIG. 7 that includes differentials for all COVID variants. The left-hand column of the table contains the results of laboratory medical tests that include tests for COVID disease (e.g., antigen test, molecular test, antibody test), and laboratory medical examinations required to obtain various patient's biochemical and biophysical data in relation to the symptoms of major COVID variants. Symptoms of COVID variants are variable, but often include fever, cough, headache, fatigue, breathing difficulties, and loss of smell and taste. The severity of mutated COVID variants varies and symptoms of mutated COVID variants are variable. Common symptoms include headache, loss of smell and taste, nasal congestion and a runny nose, a cough, muscle pain, a sore throat, a fever, diarrhea, and breathing difficulties. People with the same infection may have different symptoms, and their symptoms may change over time.

Laboratory medical tests listed in the left-hand column of the table include reverse transcription polymerase chain reaction (RT-PCR) test, nucleic acid test, serological test, molecular test CRISPR, isothermal nucleic acid amplification (INAA), digital polymerase chain reaction (DPCR), microarray analysis, next-generation sequencing (NGS), antigen tests for antigen proteins, rapid diagnostic test (RDT), enzyme-linked immunosorbent assay test (ELISA), neutralization assay, chemiluminescent immunoassay (CI). Samples for these tests can be obtained by various methods, including a nasopharyngeal swab, sputum (coughed up material), throat swabs, deep airway material collected via a suction catheter, saliva, any bodily fluid, thyroid function, genetic testing, skin testing, use of antibodies, whole blood, blood plasma, serum samples etc. Laboratory medical examinations include chest CT scans, checking for an elevated body temperature, and checking for low blood oxygen levels.

The left-hand column of the table further contains names of symptoms and diseases, for which the patient's biochemical and biophysical data is gathered by a plurality of sensors (including biosensors) for detecting respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

It should be obvious to those skilled in the art which sensors can be used for each individual symptom, and therefore, it is pointless to list all these sensors in the present invention, especially when new and enhanced sensors are continuously being introduced in medical practice (e.g., biosensors which convert a biological response into an electrical signal and combine a biological component, e.g., tissue, microorganisms, organelles, cell receptors, enzymes, antibodies, nucleic acids, etc., with a physicochemical detector). It is also obvious that as many available sensors should be used and as many laboratory medical tests, laboratory medical examinations should be run as possible to obtain maximum data. Sensors for other symptoms not mentioned above can also be used, if necessary, such as blood sugar sensors, etc.

The header of the table contains the names of the original COVID-19 virus and major COVID variants: Alpha (lineage B.1.1.7), B.1.1.7 (E484K), Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Lineage B.1.1.207, Lineage B.1.1.317, Lineage B.1.616, Lineage B.1.618 and Omicron (lineage B.1.1.529). This list is not complete and can be further expanded by adding new COVID variants discovered later.

Based on the scientific medical literature, medical guidelines provide predetermined symptom threshold values that can be used to identify major COVID variants (e.g., those listed in the header of the table). Therefore, it is possible to calculate the actual differentials (differences) between the patient data obtained from a plurality of sensors, through laboratory medical tests, laboratory medical examinations (listed in the left-hand column of the table), and the predetermined symptom threshold values that would help to identify major COVID variants. These differentials are added to the table, so the table contains differentials that represent differences between the patient data obtained and predetermined symptom threshold values for the major COVID variants. This is a deviation from the conventional medical practice, wherein the difference is calculated between the data of an ill patient and the data of a healthy person.

Another point is that the differentials mentioned in the present invention may be negative. For example, a healthy person's temperature is about 36.6° C., while the temperature indicative of the Delta variant is 40° C. According to conventional medical practice, if the patient's temperature is 38° C., for instance, then the differential is not calculated, as the 40° C. threshold value hasn't been reached. Further diagnosis of the Delta variant is not carried out. According to the present, differentials are always calculated, and the diagnosis is carried out, even if the difference is negative (−2° C.). It is possible that differentials of other symptoms all point to the fact the patient has Delta variant, while the insufficiently high temperature is due to the patient's individual physiological parameters.

Therefore, the data in the table includes differentials, both positive and negative, between the values of the patient's biochemical and biophysical data and predetermined symptom threshold values for the original COVID-19 virus strain and all major COVID variants. These differentials may be represented by actual values (figures), e.g., 23, 5, −6, as well as by Latin letters, e.g., in alphabetical order, indicating increasing or decreasing difference from the threshold value (L, B, A). Also, differentials can be represented by a percentage of the threshold value, e.g., 10%, 2%, −3%, or by the plus and minus signs (as shown in FIG. 8), where the plus sign represents the fact the threshold value for a given COVID variant was exceeded, and the minus sign represents the fact it was not. In the same vein, similarly, a binary code can be used, where 1s represent the fact the threshold value for a given COVID variant was exceeded, and 0s represent the fact it was not. Other symbols and designations can be used as well, e.g., color codes, etc.

The set of differentials in table 701 is analyzed by using the methods of cluster analysis 704 for calculating the statistics of values of differentials within the table. In an aspect, the differentials within the table of differentials 701 are combined into multiple groups of differentials 707 based on the differences in the differentials. The differentials not included in the groups 707 are not taken into account in the further analysis of the set of differentials. Based on the multiple groups of differentials 707 detected, the set of differentials is analyzed to detect correlations indicative of relationships between the groups of the differentials.

In another aspect, the set of differentials 701 is also complemented with the set of patient's biochemical and biophysical data obtained, the set of predetermined symptom threshold values for all major COVID variants, the set of patient's individual physiological parameters, the set of patient's diseases that accompany COVID-19, and the set of additional patient data. In all these sets, statistics are calculated and correlations 706 are detected using the method of combinatorial statistical analysis 702 and the methods of cluster analysis 704 for comparing values of data and finding correlations within values of the data. It should be obvious to those skilled in the art that other statistical methods can be used as well.

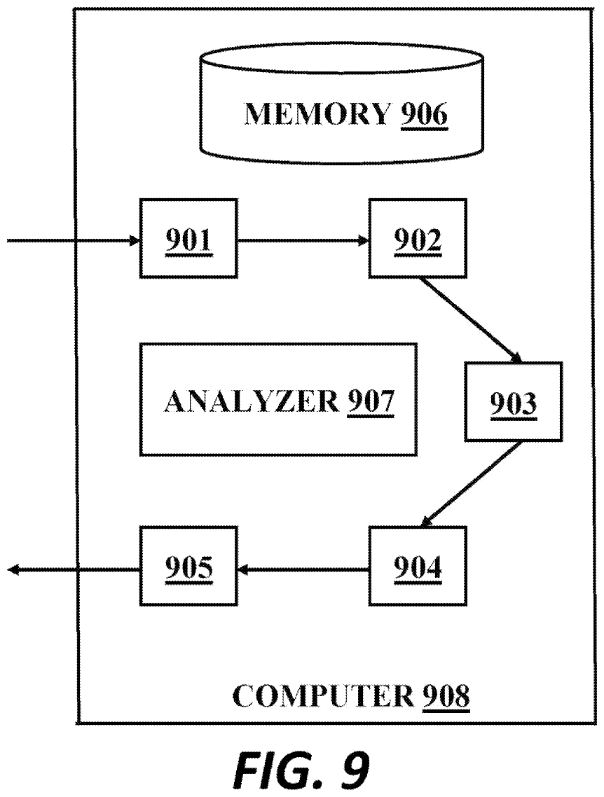

FIG. 9 is a diagram illustrating a hardware system for implementing the method of combinatorial statistical analysis 702 of FIG. 7. The aim of the method of combinatorial statistical analysis is to allow the user to enter lists of the numbers and/or terms in double or triple combinations, and compare it with each other to find specific correlations indicative of the mathematical (statistical) or logical relationships between the values.

The system of FIG. 9 consists of 5 software components: 1) unit 901 for lists selection (or for at least one database selection) allowing the user to enter the values and/or terms of interest in a combinatorial fashion in different lists, 2) a co-occurrence frequency retrieval unit 902 wherein the unit extracts the co-occurrence and separately occurring statistics of the values and/or terms of interest in a combinatorial fashion from the databases, 3) a normalization unit 903 wherein the ratio of co-occurrence statistics of the values and/or terms to the separately occurring statistics are calculated using various formulas, 4) data integration unit 904 where the normalized data is integrated on a matrix, 5) the display unit 905 where the data is displayed to the end-user in a graphical format.

The units 901-905 implemented in the central memory 906 of a computer 908 or on one of its storage units from a storage medium, for example, a CD-ROM, or through the transmission of a data feed. Actions on values and data are implemented through the analyzer 907 that is loaded into the computer 908.

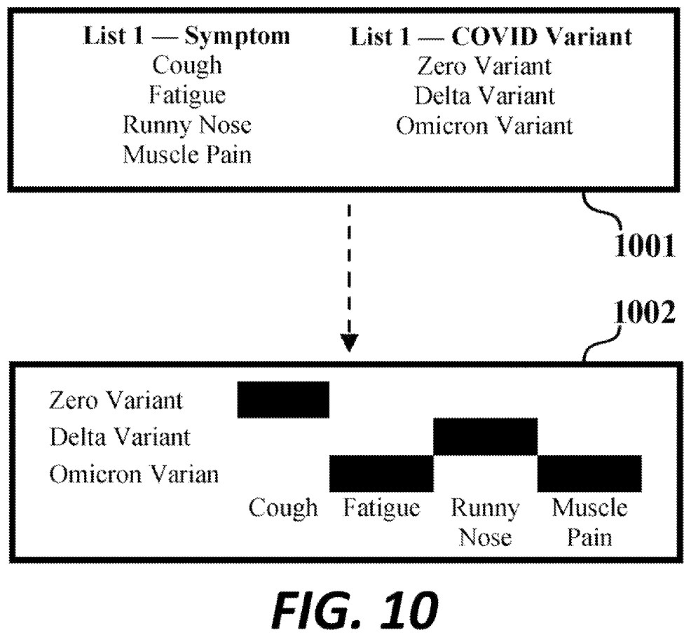

The method of combinatorial statistical analysis functions in the following fashion: 1) at least one database is chosen by the user, 2) the values and/or terms of interest are entered by the user in at least two lists with respect to the order of interest (as shown on 1001 in FIG. 10), 3) determination of co-occurrence as well as separately occurring frequencies for the values and/or terms of different lists in a combinatorial fashion, 4) data normalization via ratio calculation of the co-occurrence statistics to the separately occurring statistics using different ratio formulas, 5) elimination of errors and data normalization according to the normalization step, 6) graphical display of the results (as shown on 1002 in FIG. 10) to the user.

The method of combinatorial statistical analysis allows the user to search for symptoms of major COVID variants, and to read and interpret the results in the following fashion: 1) the selection of the main database, 2) entrance of the values of differentials and values of patient data obtained into list 1 and list 2, 3) determination of the occurring frequencies of values in list 1 and list 2 separately on the database, 4) determination of the co-occurrence of frequencies of values in list 1 and values in list 2 in a combinatorial fashion, 5) ratio normalization of the values of frequencies of list 1 and list 2 in a combinatorial fashion, 6) error elimination with respect to results of the normalization, 7) integration of the obtained data on a matrix and displaying to the end-user using the color code. The results will show the user which symptoms of major COVID variants are probable based on the statistics of the data in table 701.

Or, for example, we can compare and analyze the set of differentials and the set of predetermined symptom threshold values for major COVID variants to detect diseases of major COVID variants. For this we will use the method of combinatorial statistical analysis 702 to search for COVID disease in the following fashion: 1) the selection of the main database, 2) entrance of the values of differentials and predetermined symptom threshold values into list 1 and list 2 (as below using the entrance unit), 3) determination of the occurring frequencies of values in the list 1 and list 2 separately on the database, 4) determination of the co-occurrence frequencies of values in list 1 and values in list 2 in a combinatorial fashion, 5) ratio normalization of the values of frequencies of list 1 and list 2 in a combinatorial fashion, 6) error elimination with respect to results of the normalization, 7) integration of the obtained data on a matrix and displaying to the end-user using the color code. The results will show the user which diseases of major COVID variants are probable based on the statistics of the data in table 701.

The display 1002 of FIG. 10 illustrates an example of the result of using the method of combinatorial statistical analysis. The example 1002 shows detected COVID variants using a color code. It shows the correlations of the symptoms of major COVID variants to the diseases of major COVID variants that user has previously entered into two lists 1001 (or other information of interest, e.g., the correlations of the values of differentials to the values of patient data obtained, or the correlations of the values of differentials to predetermined symptom threshold values, etc.)

The method of combinatorial statistical analysis 702 is used to compare pairs of datasets in order to calculate statistics and find correlations 706 that are the mathematical (statistical) or logical relationships between the values among the following fifteen pairs of datasets: 1) differentials and patient's biochemical and biophysical data, 2) differentials and predetermined symptom threshold values for major COVID variants, 3) differentials and patient's individual physiological parameters, 4) differentials and diseases that accompany COVID-19, 5) differentials and additional patient data, 6) patient's biochemical and biophysical data and predetermined symptom threshold values for major COVID variants, 7) patient's biochemical and biophysical data and patient's individual physiological parameters, 8) patient's biochemical and biophysical data and diseases that accompany COVID-19, 9) patient's biochemical and biophysical data and additional patient data, 10) predetermined symptom threshold values for major COVID variants and patient's individual physiological parameters, 11) predetermined symptom threshold values for major COVID variants and diseases that accompany COVID-19, 12) predetermined symptom threshold values for major COVID variants and additional patient data, 13) patient's individual physiological parameters and diseases that accompany COVID-19, 14) patient's individual physiological parameters and additional patient data, 15) diseases that accompany COVID-19 and additional patient data.

In order to detect correlations 706 in a large array of data, e.g., comparing three or more datasets and finding correlations there, the mathematical method of dense network of curves 703 is used, for instance, to detect correlations between differentials, patient's biochemical and biophysical data, patient's individual physiological parameters, patient's diseases that accompany COVID-19, and additional patient data. The mathematical method of dense network of curves allows for a superior level of analysis of the aforementioned data, both qualitatively and quantitatively.

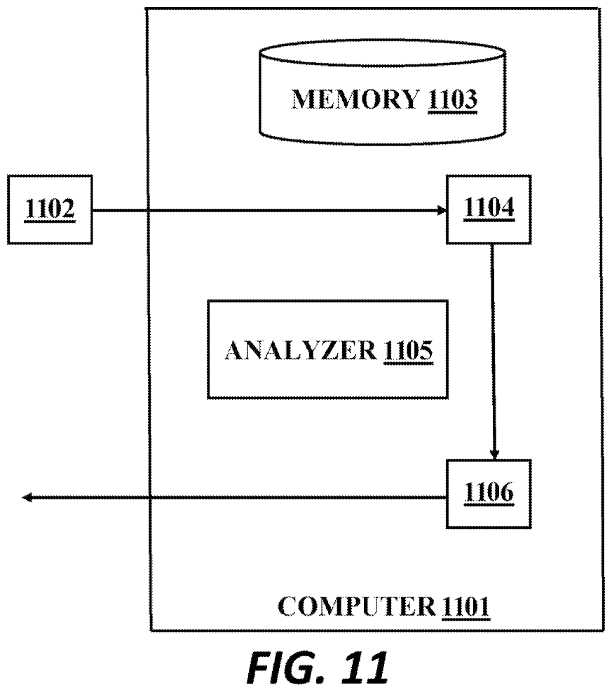

FIG. 11 is a diagram illustrating a hardware system for implementing the mathematical method of dense network of curves 703 of FIG. 7. The system of FIG. 11 includes the computer 1101, the chronological set of numerical values 1102, the central memory 1103, the unit of data entry 1104, the analyzer 1105, the display unit 1106. The chronological set of values 1102 is entered into the unit of data entry 1104 stored in the central memory 1103 of a computer 1101 or on one of its storage units from a storage medium, for example, a CD-ROM, or through the transmission of a data feed. The numerical values of the chronological set 1102 are used in the system in order to construct a dense network of curves constituting the topological structure of the set. Operations on the numerical values of the chronological set 1102 according to the mathematical formula (that be given below) are implemented through the analyzer 1105 that is loaded into the computer 1101.

The system is used to construct a dense network of curves constructed mathematically from numerical data of the chronological set 1102 (e.g., the values of the patient's biochemical and biophysical data or the values of the differentials) and defined by a primary parameter (the number of data points used) and a secondary parameter (the scale parameter). The secondary parameter (the scale parameter) can be the interval of time separating two consecutive data points, for example, minutes, hours, or days, since the onset of the illness or the patient was in quarantine.

Other types of intervals can also be used. For differentials, for example, the scale can be expressed in terms of the number of same values of differentials to one patient or the number of same values of differentials to one major COVID variant. Or, for example, a dense network of curves can be constructed mathematically from the values of differentials of the chronological set 1102 and defined by a primary parameter (the number of patients with ischemic heart disease and tuberculosis) and a secondary parameter (the duration of the patient's illness or having immunity).

The system can use any of the following regressions: 1) regression of order zero, otherwise known as average, 2) first order regression, otherwise known as linear regression, 3) second order regression, otherwise known as quadratic regression, 4) regression of order greater than 2. The curves of this network belong to one of the following categories: 1) moving regression (MR) of degree zero, known as the moving average (MA), 2) MR of the first degree, known as the moving linear regression (MLR), 3) MR of the second degree, which we will call the moving quadratic regression (MQR), 4) MR of the kth degree, which we will call the moving k regression (MKR).

The present invention is based on the utilization of a dense network of MRs corresponding to a large set of values of the primary parameter, chosen according to defined criteria because in this case-characteristic figures appear strikingly on the monitor of a computer 1101. The network described in what follows is composed of MLRs. It is on the presence of these characteristic figures within the dense network that rests the ability to the analysis of the data and obtain precise and reliable information. The method can also use adjusted data, for example, averaged or weighted data.

The necessary conditions under which the characteristic figures appear in the network are the following: 1) the network must contain a large number of MLRs, greater than about 50, 2) the set of the values of the primary parameter must extend over a sufficiently large range, 3) the distribution of the values of the primary parameter must be such that the corresponding network has a uniform density on average. In practice, criterion 3 is satisfied when the values of the primary parameter constituting the set grow slowly and uniformly. Furthermore, if wished, one can slightly modify the density, for example, by making the network denser for smaller values of the primary parameter.

The algebraic formula used in the present invention is:

n k=n1+(k−1)a+k(k−1)N(N−1)[n N−n1−(N−1)a]

where:
k={1, . . . N},
N is the number of curves in the network,
n₁ is the first term of the set,
n_{N} is the N^{th} term of the set,
a is the interval between n₁ and n₂.

Taking N=100, n₁=8, n_{N}=1502, and a=8 as an example, one obtains for the primary parameter the following set of values: {8, 16, 24, 33, 41, 50, 59, 68, . . . , 1351, 1372, 1393, 1415, 1436, 1458, 1480, 1502}. This set of values generates a network of 100 MLRs which has a uniform density on average and extends over a large range. The characteristic figures seen on the monitor of the computer belong to one of the following two types: 1) cord, and 2) envelopes.

A cord is a pronounced condensation of curves that stands out from a less dense background of curves of the network. An envelope outlines the boundary of a group of curves of the network. A characteristic figure attracts or repels the representative curve of the data, depending on its type, its shape and its relative position to the representative curve of the data. The more marked the characteristic figure, the stronger the attraction or the repulsion.

The analysis of the data requires the examination of the ensemble of the cords and envelopes and the representative curve of the data up to a given moment, over a sufficiently large interval of consecutive data points. An interval is considered sufficiently large when it contains a peripheral characteristic figure at the top of the network exhibiting a convex upward turning point and another one at the bottom exhibiting a convex downward turning point.

The ensemble of the cords and envelopes and the representative curve of the data observed over a sufficiently large interval are referred to as a spatial configuration. Qualitative and quantitative indications are obtained from a spatial configuration by determining which characteristic figures specifically attract and which characteristic figures specifically repel the representative curve of the data, and this is achieved through the examination of numerous and varied spatial configurations.

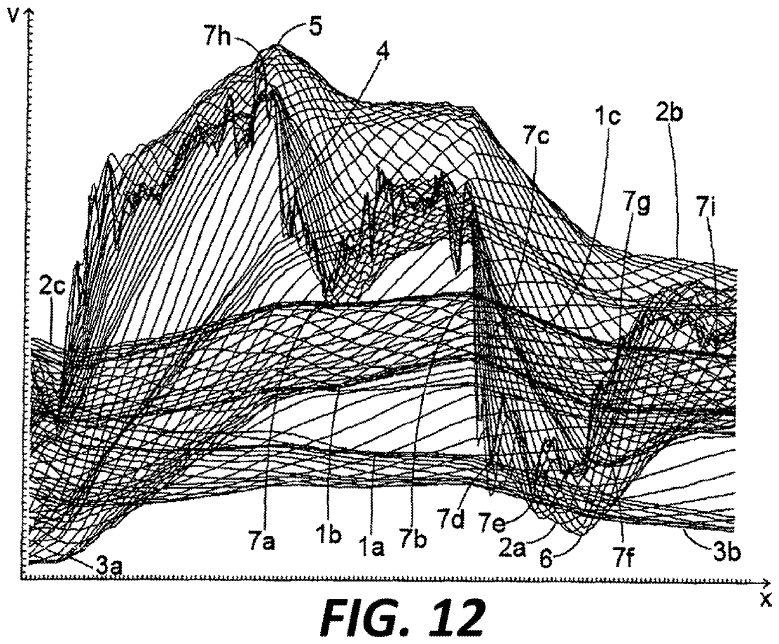

FIG. 12 illustrates a graphical example of using the mathematical method of dense network of curves 703 of FIG. 7 in which characteristic figures and spatial configurations appear. In FIG. 12, which represents a network of one hundred and fifty curves based on linear regressions calculated by the formula described above, one can see characteristic figures containing cords 1a, 1b, 1c, envelopes 2a, 2b, 2c, mixed figures (which is both a cord and an envelope) 3a, 3b and the representative curve of the set of values 4, in the form of a continuous curve.

The network contains on the upper part a peripheral characteristic figure presenting a maximum 5 and on the lower part a peripheral characteristic figure presenting a minimum 6. A characteristic figure will attract-repulse the representative curve of the chronological set of values according to its type, its shape, and its position in relation to the representative curve. For example, it is, at abscissa x0, the “attractive-repulsive” effect of the characteristic figures on the representative curve of the chronological set of values, without figure-crossing 7a, 7d, 7e, 7h, 7i and with figure-crossing 7b, 7c, 7f, 7g.

As shown in FIG. 7 by using the method of combinatorial statistical analysis 702, the mathematical method of dense network of curves 703, the methods of cluster analysis 704, it is possible to analyze all data stored in the databases on the server 709 in the system of the present invention in order to find correlations 706. Also, the method of combinatorial statistical analysis 702, the mathematical method of dense network of curves 703, the methods of cluster analysis 704 can be employed independently as machine learning techniques using the aforementioned databases on the server 709 to predict new variants of COVID-19, or create novel learning models for detecting new COVID variants.

The methods 702-705 disclosed above are used to calculate statistics, and detect and analyze correlations 706 that are the mathematical or logical relationships between the values in the following data: 1) biochemical and biophysical data obtained from sensors and biosensors (for detecting cough, sputum, shortness of breath, fever, anosmia, ageusia, nasal congestion, runny nose, sore throat, muscle pain, joint pain, headache, fatigue, abdominal pain, vomiting, diarrhea, diabetes, lung diseases, cardiovascular diseases, ischemia, hypertension), 2) biochemical and biophysical data obtained from laboratory medical examinations (chest CT scans, checking for elevated body temperature, checking for low blood oxygen level), 3) biochemical and biophysical data obtained from laboratory medical tests (the reverse transcription polymerase chain reaction test, nucleic acid test, serological test, molecular test CRISPR, isothermal nucleic acid amplification, digital polymerase chain reaction, microarray analysis, next-generation sequencing, antigen tests for antigen proteins, rapid diagnostic test, enzyme-linked immunosorbent assay test, neutralization assay, chemiluminescent immunoassay), 4) patient's individual data (whether they have tuberculosis, diabetes, pregnancy, severe immunosuppression, lymphoma, oncological diseases, ulcers, ischemic heart diseases, cardiovascular pathologies, nervous diseases, as well as their sex, age, height, weight, ethnicity, area of living, quarantine stay length, etc.)

The method of combinatorial statistical analysis 702, the mathematical method of dense network of curves 703, the methods of cluster analysis 704, the machine learning techniques 705 can be used to detect correlations 706 in this data in order to diagnose the patient's viral disease, identify the major COVID variant that is closely related to the patient's disease, find differences between diseases caused by the major COVID variants, update and revise predetermined symptom threshold values for the major COVID variants, predict the individual traits of the course of the patient's viral disease, detect individual traits of the patient's diseases that accompany COVID-19, detect the post-COVID syndrome of the patient. Detected correlations that are the mathematical or logical relationships between the values can then be used to create machine learning techniques for detecting new COVID variants.

The method of combinatorial statistical analysis 702 compares and analyze the values of biochemical and biophysical data obtained from sensors and through laboratory medical tests, laboratory medical examinations with predetermined symptom threshold values indicating to any of the major COVID variants: Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc. The data is included in lists 1 and 2 respectively, and statistics are calculated using the combinatorial approach.

In cases when a sensor reading or test result exceeds the predetermined symptom threshold values, the resulting differentials will be positive. All major COVID variants are ranked according to the total number of positive differentials they have, from the higher total to the lower total. The top-ranked COVID variant, which has more positive differentials than other variants, will be the patient's diagnosed viral disease of major COVID variant (the corresponding diagnosis is provided), and the next COVID variant will be the closely related major COVID variant, which is the closest to the patient's diagnosed viral disease (the diagnosed major COVID variant that has infected the patient).

When the mathematical method of dense network of curves 703 is used to analyze all data stored in the databases on the server 709 in the system of the present invention and interpret results, the primary parameter includes both values of differentials and predetermined symptom threshold values for major COVID variants, and the secondary parameter includes the values of the patient's biochemical and biophysical data, which are periodically updated. The resulting cords and envelopes for the representative curve of the data will show the diagnosed major COVID variant (i.e., the data forming the cord or envelope are located closer to the representative curve of the data) and the closely related major COVID variant (i.e., the data forming the characteristic figure are located further away from the representative curve of the data).

The viral disease of a major COVID variant and the closely related major COVID variant can be detected separately using the method of combinatorial statistical analysis 702, the mathematical method of dense network of curves 703, the methods of cluster analysis 704. Then all detected major COVID variants can be summed up and ranked using both methods, and the top two COVID variants may be interpreted as the patient's viral disease of major COVID variant, and as the major COVID variant that is closely related to the patient's disease respectively.

Using the method of combinatorial statistical analysis 702 to calculate statistics for the values of differentials for all major COVID variants, and the values of differentials for the original COVID-19 virus strain, which are included in lists 1 and 2 respectively, the probability of the patient being infected by a major COVID variant is calculated, and the differences between this viral disease and the original COVID-19 virus strain are determined, in case the values of differentials of the diagnosed disease do not match the values of differentials or predetermined symptom threshold values for the original COVID-19 virus strain.

In the same way, individual traits of the viral disease course are determined, wherein all values of differentials and values of patient's biochemical and biophysical data obtained from sensors and through laboratory medical tests, laboratory medical examinations are included in lists 1 and 2 respectively, and the biochemical and biophysical data are periodically updated. By calculating statistics for differentials and biochemical and biophysical data obtained over the course of the patient's viral disease using the combinatorial method, it is possible to see the progress of the viral disease. For example, if the values of differentials increase over time, then the disease is intensifying. Conversely, if the values of differentials decrease over time, then the disease is abating.

The resulting statistical correlations 706 may be uploaded into the method of combinatorial statistical analysis 702 again and compared, for example, with the patient's individual physiological parameters and additional patient data. Matches with certain patient's individual physiological parameters found therein might show individual traits of the patient's disease course. If the resulting correlations 706 are compared with the diseases that accompany COVID-19, then the combinatorial method might show individual traits of the patient's diseases that accompany COVID-19, as well as their possible post-COVID syndrome.

Also, in order to detect correlations 706, the mathematical method of dense network of curves 703 is used, wherein the primary parameter includes both values of differentials and predetermined symptom threshold values for major COVID variants, and the secondary parameter includes statistical data of major COVID variants by sex, age, and region. The resulting spatial configuration represented by cords and envelopes and applied to a representative curve of the data will show the differences between major COVID variants, in case some characteristic figures will be detected that can be compared using the primary parameter and the secondary parameter data.

So, if the primary parameter includes both values of differentials and predetermined symptom threshold values for major COVID variants, the secondary parameter includes statistical data of major COVID variants by sex, age, and region, then the characteristic figures in the spatial configuration might point out predetermined symptom threshold values to be updated, in case the characteristic figures show much difference in their secondary parameters and/or their positions in relation to the representative curve of the data.

When the mathematical method of dense network of curves 703 is used to determine the post-COVID syndrome, the primary parameter includes both the data about the patient's diseases that accompany COVID-19 and the patient's individual physiological parameters, and the secondary parameter includes both the values of differentials and the values of patient's biochemical and biophysical data. The characteristic figures in the spatial configuration might point out correlations between the data that are used to generate the characteristic figures. The values of the primary parameter for the characteristic figures will show the corresponding accompanying diseases, which, together with the patient's viral disease diagnosed as a major COVID variant, can be used to determine a possible post-COVID syndrome.

Alternatively, the primary parameter may include both the data about the patient's diseases that accompany COVID-19 and the patient's individual physiological parameters, and the secondary parameter includes the values of biochemical and biophysical data that are updated periodically. The characteristic figures in the spatial configuration might point out correlations 706 that may be used to determine the individual traits of the course of the patient's diseases that accompany COVID-19, based on the primary parameter with the characteristic figure, in case the data of the secondary parameter for the same characteristic figure change faster (meaning that the accompanying disease is intensifying) or slower (meaning that the accompanying disease is abating). Also, both the primary parameter and the secondary parameter may include the data from any of the detected correlations 706, which will be analyzed and interpreted again using the mathematical method of dense network of curves 703.

It should be obvious to those skilled in the art that the data stored in the databases on the server 709 can be analyzed using different mathematical methods. For example, the cluster analysis 704 can be used for this. Cluster analysis is the task of grouping a set of objects in such a way that objects in the same group (called a cluster) are more similar to each other than to those in other groups (clusters). The cluster analysis 704 can use the differences in the values of the all data stored in the databases on the server 709 in the system of the present invention to define multiple groups of the values and to find correlations 706 in each group. For example, the cluster analysis uses the differences in the differentials within the table 701 to define multiple groups of the differentials 707 and to find correlations 706 in each group of the differentials.

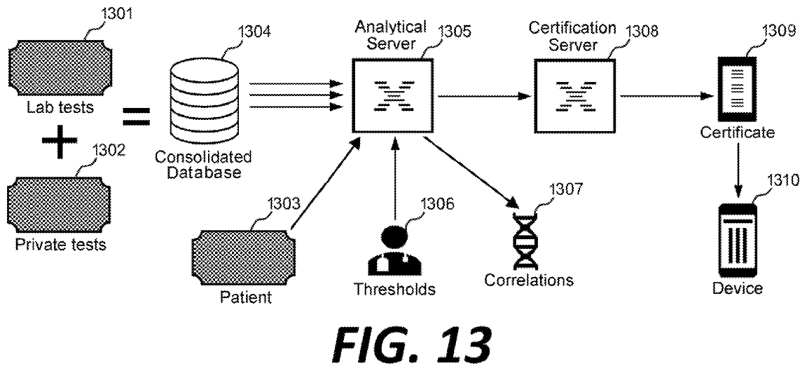

FIG. 13 is a diagram illustrating an example of detecting new COVID variants using medical testing of the present invention. Components 1301-1304 of the system represent a “testing” phase, components 1305-1307 of the system represent an “analysis” phase, and components 1308-1310 represent a “realization” phase of giving the patient a certificate.

In the “testing” phase, the patient's antibody data through medical tests can be collected in real-time. A variety of manual and/or automated medical testing can transmit a patient's antibody data to the system. The all patient data could include, but is not limited to, manual and automatic laboratory medical test 1301 (e.g., antigen test, molecular test, antibody test) in the medical institutions, private medical test 1302 (e.g., private testing) and patient's individual data 1303 (e.g., the patient's individual physiological parameters, including diseases that accompany COVID-19).

The laboratory medical tests 1301, private medical tests 1302, and the patient's individual data 1303 can be used to collect data on a variety of patients. The antibody data can be collected from a medical tests 1301 and 1302 in real-time, and/or from already administered tests. The types of patient's biological information that can be used for collection of the antibody data can include but is not limited to, whole blood, blood plasma, serum samples, etc.

The patient's antibody data can be collected through, e.g., manual input into the system, wireless computer protocols, LIS servers, HL7 diagnostic protocols, HIPAA compliant database queries, batch processing from medical records, any other means of digital entry, etc. All antibody data obtained through medical tests 1301 and 1302 are combined into a consolidated database 1304.

A consolidated database 1304 maintaining patient's antibody data is coupled to the analytical server 1305 and is updated in real-time. Configurable instructions for analysis of the data (partially or fully incorporated in the analytical server 1305) having differentials and correlation calculations logic programmed therein can receive as input the patient's antibody data from consolidated database 1304, the patient's individual data 1303, the predetermined symptom threshold values 1306, and detect the differentials and correlations 1307.

An analytical server 1305 comprises a set of instructions for calculating differentials (positive or negative) between the values of the patient's antibody data saved in consolidated database 1304 and the predetermined IGM and IGG antibody threshold values 1306 for the original COVID-19 virus strain (the first SARS-CoV-2 virus strain), calculating differentials (positive or negative) between the values of the patient's antibody data saved in consolidated database 1304 and the predetermined IGM and IGG antibody threshold values 1306 for the major COVID variant (the second SARS-CoV-2 virus strain), creating the set of differentials, analyzing the set to detect correlations 1307 that are the mathematical and/or logical relationships between the values within the set, diagnosing a COVID disease in the event at least one detected correlation 1307 indicates that the patient has contracted the COVID disease, getting a test result indicating the presence or absence of COVID disease.

In another embodiment of the present invention, an analytical server 1305 comprises a set of instructions for calculating differentials (positive or negative) between the values of the patient's antibody data saved in consolidated database 1304 and the predetermined IGM and IGG antibody threshold values 1306 for the original COVID-19 virus strain (the first SARS-CoV-2 virus strain), detecting a major COVID variant (the second SARS-CoV-2 virus strain) that is closely related to the patient's disease based on a correspondence of its symptoms to the differentials, calculating differentials (positive or negative) between the values of the patient's antibody data saved in consolidated database 1304 and the predetermined IGM and IGG antibody threshold values 1306 for the closely related major COVID variant (the second SARS-CoV-2 virus strain), creating the set of differentials, analyzing the set to detect correlations 1307 that are the mathematical or logical relationships between the values within the set, diagnosing a COVID disease in the event that at least one detected correlation 1307 indicates that the patient has contracted the COVID disease, getting a test result indicating the presence or absence of COVID disease.

In an aspect, the major COVID variant (the second SARS-CoV-2 virus strain) is detected such that its symptoms correspond to a majority of the differential detected by comparing the values of the patient's antibody data saved in consolidated database 1304 to predetermined IGM and IGG antibody threshold values 1306 for the original COVID-19 virus strain (the first SARS-CoV-2 virus strain). In another aspect, the major COVID variant (the second SARS-CoV-2 virus strain) is detected such that its symptoms correspond to a minority of the differentials detected by comparing the values of the patient's antibody data saved in consolidated database 1304 to predetermined IGM and IGG antibody threshold values 1306 for the original COVID-19 virus strain (the first SARS-CoV-2 virus strain).

In another embodiment of the present invention, a set of instructions further comprises the step of detecting yet another major COVID variant (the third SARS-CoV-2 virus strain) based on a correspondence of its symptoms to the differentials detected by comparing the values of the patient's antibody data saved in consolidated database 1304 to predetermined IGM and IGG antibody threshold values 1306 for the closely related major COVID variant (the second SARS-CoV-2 virus strain). Then differentials (positive or negative) are calculated between the values of the patient's antibody data saved in consolidated database 1304 and the predetermined IGM and IGG antibody threshold values 1306 for yet another major COVID variant (the third SARS-CoV-2 virus strain) for further creating the set of all differentials.

In another embodiment of the present invention, a set of instructions further comprises the step of analyzing the detected correlations using cluster analysis to define the same or similar correlations and combining these correlations into a group. The cluster analysis uses the differences in the correlations detected to define multiple groups of the correlations that are same or similar. Based on the multiple groups of correlations detected, a COVID disease is diagnosed in the event that at least one detected group of correlations indicates that the patient has contracted the COVID disease and a test result indicating the presence or absence of COVID disease is received.

A certification server 1308 can receive as input the consolidated diagnosis from the analytical server 1305, and generates a patient's health certificate 1309 for each patient based on this diagnosis. The patient's health certificate 1309 comprises the representation of the biometric sample of the patient. The biometric sample is one or more of a thumbprint set recorded from the patient, a retina scan recorded from the patient, and a DNA sample obtained from the patient and analyzed, etc. The patient's health certificate 1309 includes a code (i.e., a QR code) capable of being scanned to display the health certificate on user interface at the electronic device 1310.

The patient's health certificate 1309 provides the patient's disease, the test result for the patient indicating the presence or absence of COVID disease (e.g., antigen test result, molecular test result, antibody test result), the viral risk score, probability of being infected by a new COVID variant, individual differences between the patient's diagnosed disease and the original COVID-19 virus strain, the major COVID variant that is closely related to the patient's disease, the most significant patient's diseases that accompany COVID-19, possible individual traits of the patient's disease course, the general disease statistics, the projected post-COVID syndrome for the patient.

The system can receive as input at the certification server 1308 the differentials and correlations 1307 from the analytical server 1305 in real-time to assist in generation of the diagnosis for the patient. Such the differentials and correlations 1307 can include, e.g., medical guidelines on predetermined symptom threshold values for all major COVID variants. The differentials are calculated between the values of the patient's antibody data at the consolidated database 1304 and predetermined symptom threshold values 1306 for all major COVID variants to define the patient's disease of major COVID variant.

The differentials and correlations 1307 can be updated in real-time as a set of rules for the probability of being infected by a new COVID variant based on the medical guidelines. Operation of the certification server 1308 is thereby dynamic based on ongoing changes in the differentials and correlations 1307, as well as updated medical tests 1301 and 1302 for the patient received at the analytical server 1305 in real-time.

In addition to using the patient's antibody data saved in consolidated database 1304 and the patient's individual data 1303 to analyze and determine the diagnosis for the patient and the patient's health certificate 1309, the system can receive as input an aggregate from other systems to assist in the analysis of the COVID disease. The additional patient data can include, e.g., prior conditions, demographics, the open statistical data, combinations thereof, etc. The diagnosis can be output by the system as safety levels of the probability of being infected by a new COVID variant for the patient, e.g., with Level 1 being the safest, and each increasing level representing an additional level of risk of the COVID viral disease that infects the patient.

With respect to the major COVID variants, the analytical server 1305 can used antibody test data for the analysis, as well as patient's individual data. In one instance, the certification server 1308 analysis can be as follows: COVID disease IGG+ with IGG index>20 and no prior conditions, IGM negative with index <0.5, body temperature <37.8° C., and the certification server 1308 to output a viral disease Level 1 or safest level for the patient. In another instance, the analytical server 1305 analysis can be as follows: COVID disease IGG+ with IGG index>20 and no prior conditions, IGM negative with index <1, body temperature <37.2° C., and certification server 1308 to output a viral disease level of 2 or the next safest level for the patient. The patient's health certificate 1309 with safety level of the viral disease can be automatically updated in real-time based on additional medical tests 1301 and 1302 received by the system for the patient and/or based on updated differentials and correlations 1307, patient's individual data 1303.

The system can be initially programmed to a very high level of IGG antibodies and a very low level of IGM antibodies to denote a low COVID disease risk level. Such antibody levels can be used as predetermined symptom threshold values 1306 for analysis by the analytical server 1305. As the patient's antibody data changes and as the system learns more about the disease through the updated the differentials and correlations 1307, the diagnosis can be adjusted and applied to the existing dataset. The system can thereby continuously or substantially continuously update the risk level of the patient's COVID viral disease based on the updated the differentials and correlations 1307 and/or based on medical test data previously received by the system, reducing the need to re-test patients if the data is available.

In some embodiments of the present invention, such updating by the system can be based on the type of medical tests 1301 and 1302 taken previously by the patient. For example, a re-test may be necessary to determine the current level of antibodies of the patient. The continuously or substantially continuously updating of the medical tests 1301 and 1302 can be similarly used for any patient's antibody data being recorded and aggregated into consolidated database 1304, including patient's individual data 1303 inputs that may inform the viral disease score determination. For example, patient's individual data 1303 inputs that may affect the viral disease score determination can include place of origin and/or demographics where the COVID incidence is higher.

The certification server 1308 can be communicatively coupled to an internal API for transmission of a patient's health certificate 1309 to electronic medical records and human resources records in the medical institutions. External APIs can be communicatively coupled to the certification server 1308 to query the certification server 1308 for the patient's health certificate 1309 associated with patients. For external APIs, the system can output the necessary information based on the type of entity requesting the information. For example, the system can output to a requestor an indication that the patient is safe or not safe. As a further example, the system can output to a requestor via a graphical representation on an electronic device 1310 a patient's health certificate 1309 with the diagnosis, whether the patient is safe or not safe, and additional information regarding patient's disease of major COVID variant.

The patient's health certificate 1309 generated for each patient can be electronically stored in the certification server 1308. The generated patient's health certificate 1309 can also be tied to the person ID base used for various digital identifications of the patient. The person's ID number for each respective patient can be electronically tied to their corresponding patient's health certificate 1309. In this case, the person's ID can be used as a unique electronic element or identifier to access with subsequent queries for the patient's health certificate 1309 of the patient. For example, the patient's health certificate 1309 together with the person's ID may be employed to access for a patient to a public place with a large number of people, where permission to enter is required and where the spread of a viral infection is of great danger, for example, a stadium or an airplane.

In another embodiment of the present invention, the patient associated with the encrypted unique person ID number can be provided with a selection to allow the patient to electronically decide who has access to the information at the patient's health certificate 1309 and for what purpose. For example, the patient can selectively choose who has access to the patient's health certificate 1309 and associated information on a case-by-case basis. In another embodiment of the present invention, the patient can provide a one-time confirmation to allow all future access to the information at the patient's health certificate 1309 until the patient decides to opt-out of such access.

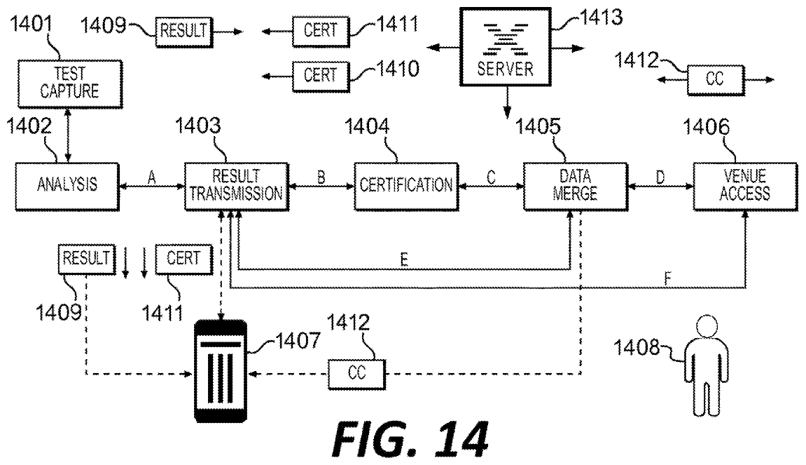

FIG. 14 is a diagram illustrating another example of detecting new COVID variants using medical testing of the present invention. The system includes a test capture component 1401, an analysis component 1402, a transmission component 1403, a certification component 1404, a data merging component 1405, a venue access component 1406. The components can be implemented by a central processing unit, server, cloud server, computer, smartphone, or other software. In FIG. 14 the components are uploaded and stored to the server 1413. In another embodiment of the present invention, the components are uploaded to the cloud server that is shared by multiple computers.

The analysis component 1402 and the transmission component 1403 communicate over interface A to cooperatively allow administration of a medical test of a user (patient) that collect antibody data, analysis of the antibody data obtained by medical tests, and generation of a test result by the test capture component 1401 and the analysis components 1402. The analysis component 1402 may supply the results of a medical test to the transmission component 1403 over the interface A.

In an aspect, the test capture component 1401, the analysis component 1402, and the transmission component 1403 may be combined in a single hardware device. In another aspect, the test capture component 1401 and the analysis component 1402 may be stand-alone hardware devices. In another aspect, some functions of the transmission component 1403 may be embodied in another component, such as smartphone 1407, or similar device, for example.

The analysis component 1402 performs the actions according to the instructions loaded into the system. The analysis component 1402 compares the values of the antibody data obtained by medical testing from the test capture component 1401 to predetermined IGM and IGG antibody threshold values for the original COVID-19 virus strain (the first SARS-CoV-2 virus strain) to calculate differentials (positive or negative), it compares the values of the antibody data obtained by medical testing from the test capture component 1401 to predetermined IGM and IGG antibody threshold values for the major COVID variant (the second SARS-CoV-2 virus strain) to calculate differentials (positive or negative), it generates the set of differential, it analyzes the set of differentials to detect correlations that are the mathematical or logical relationships between the values within the set, it runs a diagnosis based on the detected correlations that indicate the presence or absence of COVID disease, it get a test result indicating the presence or absence of COVID disease.

In another embodiment of the present invention, the analysis component 1402 compares the values of the antibody data obtained by medical testing from the test capture component 1401 to predetermined IGM and IGG antibody threshold values for the original COVID-19 virus strain (the first SARS-CoV-2 virus strain) to calculate differentials (positive or negative), it identifies a major COVID variant (the second SARS-CoV-2 virus strain) that is closely related to the disease based on a correspondence of its symptoms to the differentials, it compares the values of the antibody data obtained by medical testing from the test capture component 1401 to predetermined IGM and IGG antibody threshold values for the closely related major COVID variant (the second SARS-CoV-2 virus strain), it generates the set of differential (positive or negative), it analyzes the set of differentials to detect correlations that are the mathematical or logical relationships between the values within the set, it runs a diagnosis based on the detected correlations that indicate the presence or absence of COVID disease, it get a test result indicating the presence or absence of COVID disease.

In an aspect, the major COVID variant (the second SARS-CoV-2 virus strain) is detected such that its symptoms correspond to a majority of the differential detected by comparing the values of the antibody data obtained by medical testing from the test capture component 1401 to predetermined IGM and IGG antibody threshold values for the original COVID-19 virus strain (the first SARS-CoV-2 virus strain). In another aspect, the major COVID variant (the second SARS-CoV-2 virus strain) is detected such that its symptoms correspond to a minority of the differentials detected by comparing the values of the antibody data obtained by medical testing from the test capture component 1401 to predetermined IGM and IGG antibody threshold values for the original COVID-19 virus strain (the first SARS-CoV-2 virus strain).

In another embodiment of the present invention, a set of instructions further comprises the step of detecting yet another major COVID variant (the third SARS-CoV-2 virus strain) based on a correspondence of its symptoms to the differentials detected by comparing the values of the antibody data obtained by medical testing from the test capture component 1401 to predetermined IGM and IGG antibody threshold values for the closely related major COVID variant (the second SARS-CoV-2 virus strain). Then differentials (positive or negative) are calculated between the values of the antibody data obtained by medical testing from the test capture component 1401 and the predetermined IGM and IGG antibody threshold values for yet another major COVID variant (the third SARS-CoV-2 virus strain) for further creating the set of all differentials.

In another embodiment of the present invention, a set of instructions further comprises the step of analyzing the detected correlations using cluster analysis to define the same or similar correlations and combining these correlations into a group. The cluster analysis uses the differences in the correlations detected to define multiple groups of the correlations that are same or similar. Based on the multiple groups of correlations detected, a COVID disease is diagnosed in the event at least one detected group of correlations indicates that the patient has contracted the COVID disease and a test result indicating the presence or absence of COVID disease is received.

In an embodiment of the present invention, the test capture component 1401 and the analysis component 1402 may be implemented at a medical institution, where medical tests that collect the antibody data are carried out. The medical institution could be located at a specific venue, such as at a pharmacy or at a medical clinic. Alternatively, the medical institution could be located at the entrance to a business center or an airport terminal. The medical institution also may provide some or all the functions of the transmission component 1403. In another embodiment of the present invention, the user 1408 may employ test capture component 1401 to self-perform a medical test for a major COVID variant, such as when at home or other private settings.

The test capture component 1401 includes a medical test kit for testing a user 1408 for possible infection from a new COVID variant. Such a medical test kit may be a small, portable device configured to cooperate with the transmission component 1403. The medical test kit includes mechanisms, such as a blood sampling, nasopharyngeal swab, throat swabs, deep airway material collected via suction catheter, taking saliva, taking bodily fluid, genetic testing, skin testing, etc., to acquire a sample from the user, analyze the sample, and provide test result for major COVID variant to the transmission component 1403 over the interface A.

The transmission component 1403 may include mechanisms to control operation of the analysis component 1402. For example, when implemented in the smartphone 1407, the transmission component 1403 may include or be in communication with an application of the smartphone 1407, and the application may initiate analysis by the analysis component 1402, may provide data related to the user to the analysis component 1402, and may provide security for the test result 1409 for major COVID variant (e.g., encryption). Following any processing at the transmission component 1403, the test result 1409 for major COVID variant and any associated data may be encrypted and sent to the certification component 1404. The certificate component 1404 receives the secure test result 1409 for major COVID variant over interface B from the transmission component 1403.

In an aspect, the transmission component 1403 and the certification component 1404 may be combined into a single unit or single hardware device. In another aspect, the transmission component 1403 and the certification component 1404 may be co-located such as at a medical institution. In another aspect, the transmission component 1403 and the certification component 1404 are separated and may communicate over a wireless communications network. The transmission mechanism, when the transmission component 1403 and the certification component 1404 are not combined in a single unit, may include any suitable digital data exchange mechanism.

The certificate component 1404 functions to process a secure test result 1409 for major COVID variant and from the test result processing, generate a patient's health certificate 1410 attesting to the acceptability of the test result for one or more purposes regarding the COVID viral disease. The patient's health certificate 1410 may include the test result for COVID disease (e.g., antigen test result, molecular test result, antibody test result) as well as all information provided in the secure test result 1409 for major COVID variant. The patient's health certificate 1410 may include the date and time of sample collection, the medical test kit identification, including manufacturer and date of manufacture, test type, and a unique serial number of the medical test kit. The patient's health certificate 1410 further may include a time to live for the test result 1409 for major COVID variant, such as, for example, 24 hours, one month, etc.

The patient's health certificate 1410 also may include a quality value. The quality value may be based on the type of test and the identity of the medical test kit. The quality value further may be based on the process or modality by which the sample is collected and the test result is produced from the sample. The quality value still further may be based on the degree of security, or confidence, in the reliability of the test result. For example, a test sample collected at a medical institution and analyzed at the medical institution may have a high value. Thus a test sample collected by a medical professional at a medical clinic and processed to produce a test result by the medical professional may have the highest value. A test sample collected by user 1408 and applied to a home medical test kit may have a medium value. Other quality factors and quality rating systems may be employed.

The certificate component 1404 may provide the patient's health certificate 1410 to the component submitting the secure test result 1409 for the major COVID variant. For example, if the submitting component is the smartphone 1407 of user 1408, the certificate component 1404 may transmit the patient's health certificate 1410 to the smartphone 1407. If the test result submitting component is a medical institution, the certificate component 1404 may provide the patient's health certificate 1410 to an address input to the medical institution by the smartphone 1407, for example, an email address of the user 1408.

Alternatively, the certificate component 1404 may provide the patient's health certificate 1410 for printing at the medical institution. When printed, the patient's printed health certificate 1410 may include a tamper-proof RFID (e.g., a read-once RFID). The certificate component 1404 produces a patient's health certificate 1411 with a tamper-proof reference. The reference then may be used to look up and retrieve data such as that incorporated in the patient's health certificate 1410.

The user 1408 may employ the patient's health certificate 1411 in its digital form or in a printed form. For example, the user 1408 may provide the patient's health certificate 1411 on the user's smartphone display. In another embodiment of the present invention, the certificate component 1404, or aspects of the certificate component 1404, may be implemented in a cloud-based system. For example, the certificate component 1404 may maintain active as well as deactivated patient's health certificate in a cloud storage facility.

As noted herein, the transmission component 1403 and the certification component 1404 may be combined on a single hardware device. In an embodiment of the present invention, the certificate component 1404 may be implemented on the computer, smartphone 1407, or another smart device (such as a tablet) operated by the user 1408. In this case, the patient's health certificate 1410 is stored on the smartphone 1407, where they remain active until the expiration of the assigned time to live. When implemented on the smartphone 1407, the certificate component 1404 may be a component of the application. When implemented on a computer, the certificate component 1404 may be a component of a non-transitory computer-readable medium storing a program of instruction.

When implemented as a service (e.g., as a cloud-based service) separate from the transmission component 1403, the certificate component 1404 may transmit the patient's health certificates 1410 and 1411 to the data merging component 1405 over interface C. Such transmission may require authorization from the user 1408. In another embodiment of the present invention, the user 1408 may operate the smartphone 1407, or other smart device, to transmit the patient's health certificates 1410 and 1411 to the data merging component 1405 over interface E.

The data merging component 1405 may produce a certified certificate 1412 that the user 1408 may employ to access a specific venue by using the venue access component 1406. The specific venue is any public place with a large number of people, where a permission to enter is required and where the spread of a viral infection is of great danger, for example, a stadium or an airplane. The data merging component 1405 may produce the certified patient's health certificate 1412 by merging data from the venue access component 1406 with a patient's health certificate 1410, having acquired the patient's health certificate 1410 from the certificate component 1404 or the transmission component 1403. The data merging component 1405 may generate the certified patient's health certificate 1412 when requested or authorized to so by the transmission component 1403. The venue access component 1406 may communicate directly with the transmission component 1403 over interface F and/or with the data merging component 1405 over interface D.

The certified patient's health certificate 1412 may be produced by the venue access component 1406 based on inputs received from the test result 1409 for major COVID variant, or the transmission component 1403 and/or the data merging component 1405. In an aspect, the transmission component 1403 may provide a patient's health certificate 1410 to the venue access component 1406. In another aspect, the transmission component 1403 may provide the venue access component 1406 with authorization and a mechanism to acquire a patient's health certificate 1410 from the data merging component 1405. In another aspect, the transmission component 1403 may provide the venue access component 1406 with authorization and a mechanism to acquire a certified patient's health certificate 1412 from the data merging component 1405.

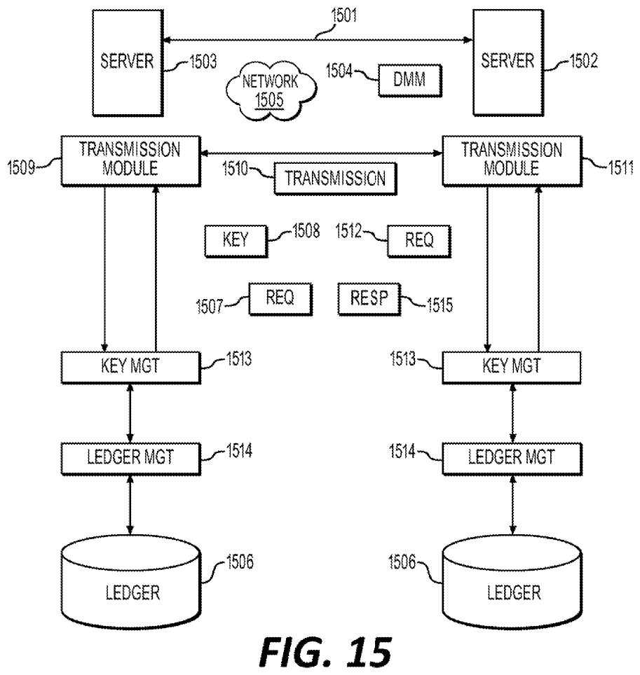

FIG. 15 is a diagram illustrating hardware and software components for implementing the present invention in greater detail. The components provide the data transmission operation of FIGS. 1-2 between servers or between the sensor and server. The data transmission operation 1501 in an embodiment of the present invention is carried out by using end-to-end encryption of the data by creating a key. This seems appropriate because some of the data transferred (for example, data on the patient's current illnesses, as well as their additional personal information) are personal data and secure methods of data transfer between servers will provide protection against possible hacking and loss of patient's personal data.

In operation, the servers 1502 and 1503 enable secure transmission of data between or on behalf of their hardware and software components through the use of data merging module (DMM) 1504 connected by network 1505. The servers 1502 and 1503 cooperate with the DMM 1504 to generate and maintain a distributed ledger 1506. The distributed ledger 1506 stores metadata associated with hardware and software components of servers 1502 and 1503. The distributed ledger 1506 implements a data structure that includes various blocks, with each block holding a batch of individual transmissions and including a timestamp indicating block inclusion in the ledger 1506.

Each server 1502 and 1503 may include a ledger management module, and a key management module. The ledger management modules manage the distributed ledger 1506. For example, the ledger management modules may propose new blocks for the distributed ledger 1506 (each proposed block containing one or more transmissions). The ledger management module further performs operations to ensure that the network node includes an updated copy of the distributed ledger 1506.

Generally, the ledger management module serves as an interface for the distributed ledger 1506. For example, the key management module may access the distributed ledger 1506 by way of the key management module. In operation, a transmission module of server 1503 may initiate a transmission with the server 1502. To execute this transmission, the transmission module of server 1503 first may query the distributed ledger 1506 to determine if a certification transmission is stored therein that would satisfy access requirements.

The distributed ledger 1506 may contain the certification transmission but not the required key. Alternatively, the distributed ledger 1506 may contain both the key and the certification transmission. Assuming only the key is not available, the server 1503 may request the server 1502 provide the required key. In response, the transmission module transmits a key request 1507 to the key management module. The key request 1507 indicates a requested transmission type (e.g., health certification (i.e., the key request 1507 is for a health credential that the transmission module requires to complete the transmission(s)).

The key management module provides a key 1508 in response to receiving the key request 1507. The key management module determines whether the key request 1507 is a valid request. The key management module accesses the distributed ledger 1506 to determine whether the key request 1507 satisfies one or more validation criterion. For example, the key management module may query the distributed ledger 1506 to determine whether the requested time duration, the requested number of transmissions, and/or the requested transmission type are permitted.

The key management module synthesizes the key 1508. The key 1508 may include a session key, a pair of keys (e.g., a public key and a private key). In some examples, the pair of keys is asymmetric or a single shared key. For example, the key management module may employ a variety of symmetric-key algorithms, such as Data Encryption Standard (DES) and Advanced Encryption Standard (AES), to generate the key 1508. Alternatively, the key management module employs a variety of public-key algorithms, such as RSA, to generate the key 1508. In an aspect, the key 1508 includes a random number. In another aspect, the key 1508 is the output of a hash function, where the hash function is a hash of the names of the entities, a time of day, and/or a random number. In yet another aspect, the key 1508 includes a credential.

The key 1508 is associated with a key identifier (ID) that identifies the key, and a validity period that indicates a time duration during which the key 1508 is valid. The validity period may be equal to requested time duration. However, if the requested time duration is greater than a threshold time duration, the validity period may be limited to the threshold time duration.

In an aspect, the key 1508 may be associated with a validity number that indicates the number of transmissions that can be completed with the key 1508. The validity number may be equal to a requested number of transmissions. However, if the requested number of transmissions is greater than a threshold number of transmissions, the validity number may be limited to the threshold number of transmissions. In another aspect, the key 1508 is associated with a validity type that indicates a transmission type that may be completed with the key request 1507. The validity type may be the same as a requested transmission type.

The transmission module 1509 may employ the key 1508 to synthesize the transmission data 1510. In an aspect, the transmission module 1509 signs the transmission data 1510 (e.g., a hash of the transmission data) with the key 1508. In another aspect, the transmission data 1510 includes encrypted data. The transmission module 1509 employs the key 1508 to encrypt the transmission data 1510. When encrypted, the transmission module 1509 transmits the transmission data 1510. The transmission module 1511 receives the transmission data 1510 and completes the transmission based on the transmission data 1510.

The transmission module 1511 may determine whether the transmission data 1510 is valid by, for example, determining whether the key 1508 employed to synthesize the transmission data 1510 is valid. As such, the transmission module 1511 transmits a validation request 1512 to the key management module 1513. In an aspect, the validation request 1512 includes the key 1508 (e.g., when the transmission data 1510 includes the key 1508). In another aspect, the validation request 1512 includes the key ID. In yet another aspect, the validation request 1512 includes only the transmission data 1510.

The key management module 1513 receives the validation request 1512 and determines whether the key 1508 employed to synthesize the transmission data 1510 is valid by, for example, querying the distributed ledger 1506 with the key 1508 and/or the key ID. The second key management module 1514 then transmits a validation response 1515 to the transmission module 1511. The validation response 1515 indicates a validity status of the key 1508. For example, the validation response 1515 may indicate the validity period, the validity number, and/or the validity type associated with the key 1508 are satisfied.

Based on the validation response 1515, transmission module 1511 employs the transmission data 1510 to complete the transmission. For example, the transmission module 1511 may complete the transmission if the validation response 1515 indicates that the transmission data 1510 was synthesized with a valid key (e.g., the key 1508 is valid). In another aspect, the transmission module 1511 may access the distributed ledger 1506 to determine whether the transmission is permitted. If the distributed ledger 1506 indicates that the transmission is permitted, the second transmission module 1511 completes the transmission.

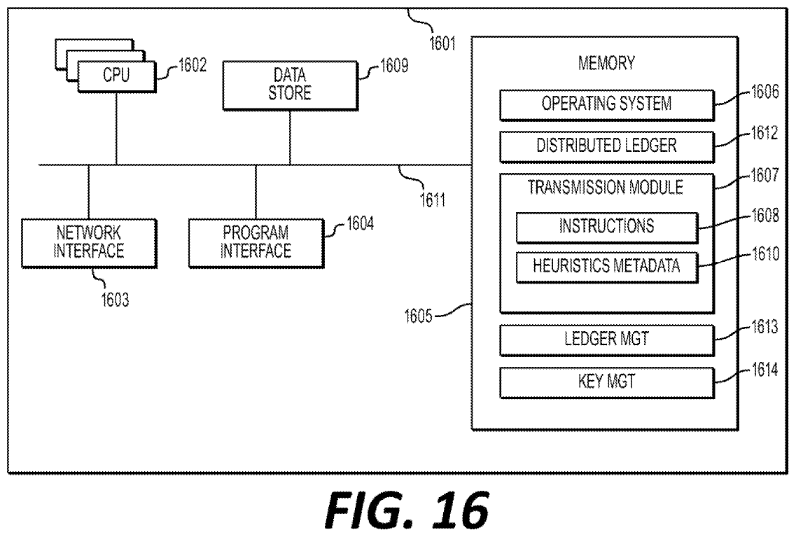

FIG. 16 illustrates components of the data merging module 1504 of FIG. 15, which is used to transmitting data between servers or between the sensor and server. The data merging module (DMM) includes server sub-system 1601. Server sub-system 1601 in turn includes one or more CPUs 1602, network interface 1603, program interface 1604, and memory 1605. Memory 1605 is a non-transitory computer-readable memory. Memory 1605 includes server operating system (OS) 1606 and transmission module 1607. Transmission module 1607 includes machine instructions 1608, which may be loaded from non-transitory computer-readable storage medium (i.e., data store) 1609, and heuristics and metadata 1610. The CPUs 1602, network interface 1603, program interface 1604, memory 1605, and data store 1609 communicate over system bus 1611. The operating system 1606 includes procedures for handling various basic system services and for performing hardware-dependent tasks.

The transmission module 1607 manages transmissions between servers or between the sensor and server. For example, the transmission module 1607 may transmit a key request to a network node within a cluster of network nodes that are configured to maintain a distributed ledger. The transmission module 1607 receives a key in response to transmitting the key request and synthesizes transmission data with the key. The transmission module 1607 transmits the transmission data to another entity. The transmission module 1607 receives transmission data, transmits a validation request to determine whether the key utilized to synthesize the transmission data is valid, receives a validation response, and utilizes the transmission data to complete a transmission if the validation response indicates that the key is valid. To that end, the transmission module 1607 includes machine instructions 1608, and heuristics and metadata 1610.

The memory 1605 and/or the data store 1609 also stores programs, modules, and data structures to enable a distributed ledger 1612, a ledger management module 1613, and a key management module 1614. The distributed ledger 1612 may be distributed over various network nodes. In some aspects, each network node stores a local copy of the distributed ledger 1612. The distributed ledger 1612 may store information regarding transmissions between servers or between the sensor and server. In some aspects, the distributed ledger 1612 stores a batch of transmissions in a block. In some aspects, each block is timestamped.

The ledger management module 1613 manages the distributed ledger 1612. For example, the ledger management module 1613 functions to ensure that the local copy of the distributed ledger 1612 is synchronized with the local copy of the distributed ledger 1612 at other network nodes. The ledger management module 1613 participates in consensus protocols associated with the distributed ledger 1612. For example, the ledger management module 1613 may propose new blocks for the distributed ledger 1612 and/or votes on block proposals received from other network nodes. To that end, the ledger management module 1613 includes machine instructions, and heuristics, and metadata.

The key management module 1614 receives a key request from an entity, determines whether the key request is valid, synthesizes a key if the key request is valid, transmits the key to the entity, and stores the key in the distributed ledger 1612. The key management module 1614 determines whether the key request is valid by determining whether one or more validation criterion stored in the distributed ledger 1612 is satisfied. For example, the key management module 1614 receives a validation request from an entity, accesses the distributed ledger 1612 to determine whether the key utilized to synthesize the transmission data is valid, and transmits a validation response that indicates the validity status of the key to the entity. To that end, the key management module 1614 includes machine instructions, heuristics, and metadata.

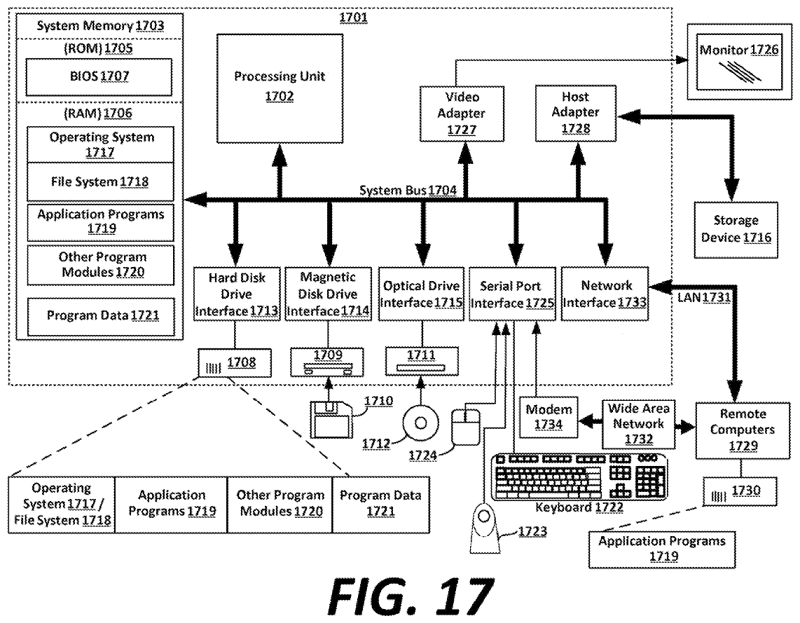

FIG. 17 is a diagram illustrating an example of a computer system for implementing the present invention. The computer system includes a general purpose computing device in the form of a host computer or a server, on which steps are performed according to the computer implemented algorithm of FIGS. 3-6 of the present invention. To execute the algorithm, a host computer or a server 1701 includes a central processing unit (CPU) 1702, a system memory 1703, and a system bus 1704 that couples various system components including the system memory to the central processing unit 1702.

The system bus 1704 may be any of several types of bus structures including a memory bus or memory controller, a peripheral bus, and a local bus using any of a variety of bus architectures. The system memory includes a read-only memory (ROM) 1705 and random access memory (RAM) 1706. A basic input/output system 1707 (BIOS), containing the basic routines that help to transfer information between the elements within the computer 1701, such as during start-up, is stored in ROM 1705.

The computer or server 1701 may further include a hard disk drive 1708 for reading from and writing to a hard disk, not shown herein, a magnetic disk drive 1709 for reading from or writing to a removable magnetic disk 1710, and an optical disk drive 1711 for reading from or writing to a removable optical disk 1712 such as a CD-ROM, DVD-ROM or other optical media. The hard disk drive 1708, magnetic disk drive 1709, and optical disk drive 1711 are connected to the system bus 1704 by a hard disk drive interface 1713, a magnetic disk drive interface 1714, and an optical drive interface 1715, respectively.

The drives and their associated computer-readable media provide non-volatile storage of computer readable instructions, data structures, program modules, and other data for the server 1701. Although the exemplary environment described herein employs a hard disk (storage device 1716), a removable magnetic disk 1710, and a removable optical disk 1712, it should be appreciated by those skilled in the art that other types of computer readable media that can store data that is accessible by a computer, such as magnetic cassettes, flash memory cards, digital video disks, Bernoulli cartridges, random access memories (RAMs), read-only memories (ROMs) and the like may also be used in the exemplary operating environment.

A number of program modules may be stored on the hard disk (storage device 1716), magnetic disk 1710, optical disk 1712, ROM 1705, or RAM 1706, including an operating system 1717 (e.g., MICROSOFT WINDOWS, LINUX, APPLE OS X or similar) The server/computer 1701 includes a file system 1718 associated with or included within the operating system 1717, such as the Windows NT™ File System (NTFS) or similar, one or more application programs 1719, other program modules 1720, and program data 1721. A user may enter commands and information into the server 1701 through input devices such as a keyboard 1722, a webcam 1723, and pointing device (e.g., a mouse) 1724. Other input devices (not shown) may include a microphone, joystick, game pad, satellite dish, scanner or the like.

These and other input devices are often connected to the central processing unit 1702 through a serial port interface 1725 that is coupled to the system bus, and they may also be connected by other interfaces, such as a parallel port, game port, or universal serial bus (USB). A monitor 1726 or other type of display device is also connected to the system bus 1704 via an interface, such as a video adapter 1727. In addition to the monitor 1726, computers typically include other peripheral output devices (not shown), such as speakers and printers. A host adapter 1728 is used to connect to the storage device 1716.

The server/computer 1701 may operate in a networked environment using logical connections to one or more remote computers 1729. The remote computer (or computers) 1729 may be another personal computer, a server, a router, a network PC, a peer device, or other common network node, and it typically includes some or all of the elements described above relative to the server 1701, although here only a memory storage device 1730 with application software 1719 is illustrated. The logical connections include a local area network (LAN) 1731 and a wide area network (WAN) 1732. Such networking environments are common in offices, enterprise-wide computer networks, Intranets, and the Internet.

In a LAN environment, the server/computer 1701 is connected to the local area network 1731 through a network interface or adapter 1733. When used in a WAN networking environment, the server 1701 typically includes a modem 1734 or other means for establishing communications over the wide area network 1732, such as the Internet. The modem 1734, which may be internal or external, is connected to the system bus 1704 via the serial port interface 1725. In a networked environment, the program modules depicted relative to the computer or server 1701, or portions thereof, may be stored in the remote memory storage device. It will be appreciated that the network connections shown are merely exemplary and other means of establishing a communications link between the computers may be used.

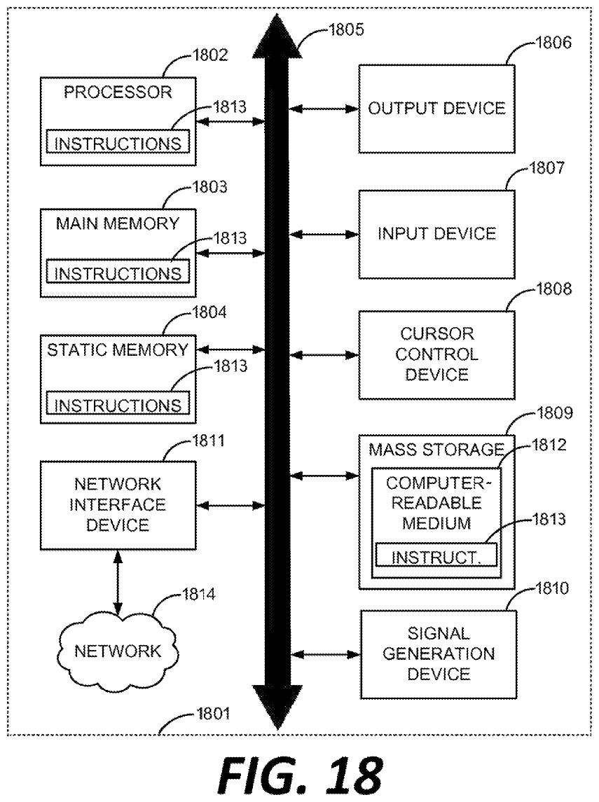

Other embodiments of computer system for implementing the present invention can be realized, as shown in FIG. 18. For example, an article of manufacture, such as a computer 1801, a memory system, a magnetic or optical disk, some other storage device, or any type of electronic device or system can include one or more processors 1802 coupled to a non-transitory computer-readable medium 1812 such as a memory (e.g., removable storage media, as well as any memory including an electrical, optical, or electromagnetic conductor) having instructions 1813 stored thereon (e.g., computer program instructions), which when executed by the one or more processors 1802 result in performing the steps of the algorithm of FIGS. 3-6 of the present invention.

The computer 1801 can take the form of a computer system having a processor 1802 coupled to a number of components directly, and/or using a bus 1805. Such components can include main memory 1803, static or non-volatile memory 1804, and mass storage 1809. Other components coupled to the processor 1802 can include an output device 1806, such as a video display, an input device 1807, such as a keyboard, a cursor control device 1808, such as a mouse, and a signal generation device 1810 (e.g., a speaker or a light emitting diode (LED)). A network interface device 1811 to couple the processor 1802 and other components to a network 1814 can also be coupled to the bus 1805.

The instructions 1813 can further be transmitted or received over the network 1814 via the network interface device utilizing any one of a number of well-known transfer protocols (e.g., HTTP). While the non-transitory computer-readable medium 1812 is shown as a single medium, the term “computer-readable medium” should be taken to include a single medium or multiple media (e.g., a centralized or distributed database, or associated caches and servers, and or a variety of storage media, such as the processor 1802 registers, memories 1803, 1804, and the storage device 1809) that store the one or more sets of instructions 1813.

Any of these elements coupled to the bus 1805 can be absent, present singly, or present in plural numbers, depending on the specific embodiment to be realized. In an example, one or more of the processor 1802, the memories 1803, 1804 storage device 1809 can each include instructions 1813 that, when executed, can cause the computer 1801 of the steps of the algorithm of FIGS. 3-6 of the present invention. In alternative embodiments, the computer 1801 operates as a standalone device or can be connected (e.g., networked) to other machines. In a networked environment, the computer 1801 can operate in the capacity of a server or a client machine in server-client network environment, or as a peer machine in a peer-to-peer (or distributed) network environment.

The computer 1801 can include a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine 1801 of the machines that individually or jointly execute a set (or multiple sets) of instructions to perform the steps of the algorithm of FIGS. 3-6 of the present invention.

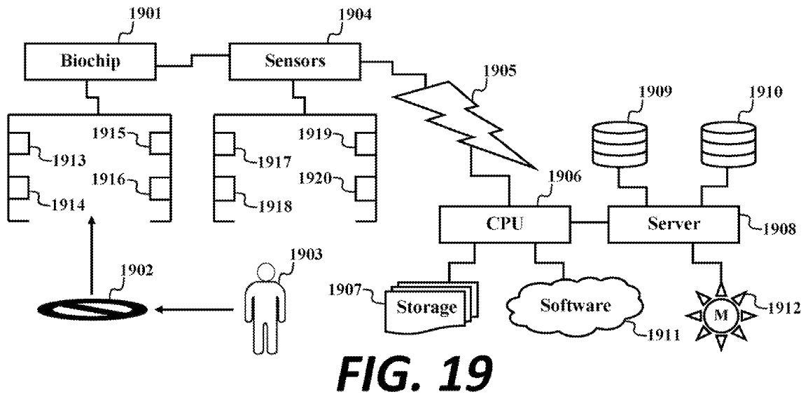

FIG. 19 is a diagram illustrating an example of a system for detecting COVID variants implemented in the device known as a “Covidometer.” The “Covidometer” of the present invention comprises a biochip 1901 collects a biological sample 1902 from the patient 1903, a plurality of sensors (e.g., a sensors panel) 1904 that gather the values of the biochemical and biophysical data from the biological sample 1902, a transmission system 1905 to transmit data gathered from the biochip 1901 and sensors 1904, a central processing unit 1906 in communication with the transmission system 1905 to collect the data gathered from the biochip 1901 and sensors 1904, a storage 1907 that receives and stores collected biological sample 1902, a server 1908 in communication with the central processing unit 1906 that comprise a database 1909 of the gathered values of the biochemical and biophysical data and a database 1910 of predetermined symptom threshold values for all COVID variants, a software application 1911 that is downloadable to and executable by the central processing unit 1906 of the “Covidometer” to cause the system to perform the steps of the algorithm of FIGS. 3-6 of the present invention, as well as means (e.g., a display monitor or printer) 1912 for outputting a result indicating a presence or absence of COVID disease in a patient 1903.

The biochip 1901 has incorporated thereon a number of biosensors 1913-1916, which are mounted thereon. The biosensors 1913-1916 collect a biological sample 1902 (e.g., antibodies, whole blood, blood plasma, serum samples, nasopharyngeal swab, throat swabs, deep airway material, saliva, any bodily fluid, skin imprint, etc.) from the patient 1903, which will be analyzed by sensors 1917-1920, which are installed on the sensors panel to gather the values of the biochemical and biophysical data from the biological sample 1902. In an aspect, the biosensors 1913-1916 work based on the use of enzymes, whole cell metabolism, ligan debinding and antibody-antigen reaction. The biosensors produce an electrical signal (e.g., electrochemical change to detect presence of antigens) detectable by the sensors 1917-1920 capable of distinguishing IgM and IgG antibodies from each other (e.g., an electrochemical immuno sensors capable of distinguishing IgM and IgG antibodies from each other).

Once the biochip 1901 has gathered a biological sample 1902 from the patient 1903 by using the biosensors 1913-1916, the biological sample 1902 will be delivered in the storage 1907 that receives and stores collected the biological sample from the patient. The purpose of the storage 1907 (e.g., the box for biological materials) is to provide for preservation of the biological sample 1902 of the patient 1903 to enable it to be reanalyzed by the “Covidometer.” The analysis of the biological sample 1902 will result in sending the values of the biochemical and biophysical data gathered from the biological sample 1902 by using the sensors 1917-1920 to a server 1908. The sensors 1917-1920 installed in the sensors panel 1904 of the “Covidometer” are replaceable so that a failure of any sensor could be addressed by a simple replacement of the sensor, allowing simple and robust connection with hardware components of the “Covidometer” by common protocols and procedures following universal standards.

The process to collect the values of the biochemical and biophysical data from the biological sample 1902 stored in the storage 1907 by using the sensors 1917-1920 may follow a process having a number of steps. At the initial stage the values of data will be retrieved from the biological sample 1902 of patient 1903. At the next stage the symptom data values representing the symptoms of patient 1903 will be determined from the values of data retrieved from the biological sample 1902. The symptoms of patient 1903 may be respiratory symptoms (cough, sputum, shortness of breath, fever, anosmia (loss of smell), ageusia (loss of taste), nasal congestion, runny nose, sore throat), musculoskeletal symptoms (muscle pain, joint pain, headache, fatigue), digestive symptoms (abdominal pain, vomiting, diarrhea), physiological diseases (diabetes, lung diseases, cardiovascular diseases, ischemia, hypertension).

The transmission system 1905 transmit the symptom data values gathered from the sensors 1917-1920, and thereafter the central processing unit 1906 in communication with the transmission system 1905 collect the data values. The central processing unit 1906 is a transmitting or monitored central processing unit which is connected through the transmission system 1905 to biosensors 1913-1916 and sensors 1917-1920. In an aspect, the transmitting or monitored central processing unit will be connected to a transmission system 1905, e.g., standard telecommunication network, and thereafter the symptom data values will be delivered to the central processing unit 1906. The data values is transferred by the sensors 1917-1920 using a secure encoded channel, and levels of encryption are applied to all data transfer. In another aspect, the “Covidometer” may further include the transmitting system central processing unit remote from the central processing unit 1906. For example, the transmitting system central processing unit may be installed on a biochip 1901.

The central processing unit 1906 is connected to a transmission system 1905, and thereafter all the symptom data values gathered by using the biosensors 1913-1916 and sensors 1917-1920 will be delivered to a server 1908. The server 1908 in communication with the central processing unit 1906 comprises the database 1909 of the gathered symptom data values and the database 1910 of predetermined symptom threshold values for all known COVID variants: Alpha (lineage B.1.1.7), B.1.1.7 with E484K, Beta (lineage B.1.351), Gamma (lineage P.1), Delta (lineage B.1.617.2), Lambda (lineage C.37), Mu (lineage B.1.621), Epsilon (lineages B.1.429, B.1.427, CAL.20C), Zeta (lineage P.2), Theta (lineage P.3), Eta (lineage B.1.525), Iota (lineage B.1.526), Kappa (lineage B.1.617.1), Omicron (lineage B.1.1.529), Lineage B.1.1.207, Lineage B.1.1.317, Lineage B.1.616, Lineage B.1.618, Brazilian variant, Centaurus variant, Deltacron variant, etc.

A software application 1911 receives information and executing by the central processing unit 1906 within the server 1908 comprised the databases 1909 and 1910, on which steps are performed according to the algorithm of FIGS. 3-6 for how to manipulate the databases 1909 and 1910 of medical data. In general case, the algorithm comprises the steps of: calculating first differentials (positive or negative) for the first COVID virus strain by comparing the values stored in the database 1909 to first predetermined symptom threshold values for the first COVID virus strain stored in the database 1910, using the first differentials to detect a second COVID virus strain with a mutated virus genome code based on a correspondence of their symptoms to the first differentials, calculating second differentials (positive or negative) for the second COVID virus strain by comparing the values stored in the database 1909 to second predetermined symptom threshold values for the second COVID virus strain stored in the database 1910, creating a set of the first and second differentials, detecting correlations within the superset, determining that the patient 1903 has the first or second COVID virus strain based on the detected correlations, outputting a result indicating a presence or absence of COVID disease in a patient 1903 by using the means 1912.

One of the ordinary skills in the art will further understand the various programming languages that can be employed to create one or more the software applications 1911 designed to implement and perform the steps of the algorithm of FIGS. 3-6 of the present invention. For example, the software applications 1911 can be structured in an object-orientated format using an object-oriented language, such as Java, C++, or one or more other languages. Alternatively, the software applications 1911 can be structured in a procedure-orientated format using a procedural language, such as assembly, C, etc.

Means 1912 outputs a result indicating a presence or absence of COVID disease in a patient 1903. Means 1912 for outputting a result are an electronic device (such as a liquid-crystal display (LCD)) or part of a device (such as the screen of a tablet) that presents the result in visual form for patient 1903. Means 1912 can be any piece of computer hardware equipment which converts information into a human-perceptible form, such as text, graphics, audio, or video. Examples of means 1912 include monitors (e.g., a computer monitor or studio monitor), speakers (e.g., computer speakers), headphones, projectors (e.g., a LED projector), printers (e.g., inkjet printers, laser printers, thermal printers, dot matrix printers), tactile displays, raille displays, terminals for outputting information (e.g., a monochromatic terminal), punched cards, etc. It should be understood that other and further modifications of the “Covidometer,” apart from those shown or suggested herein, may be made within the spirit and scope of the present invention.

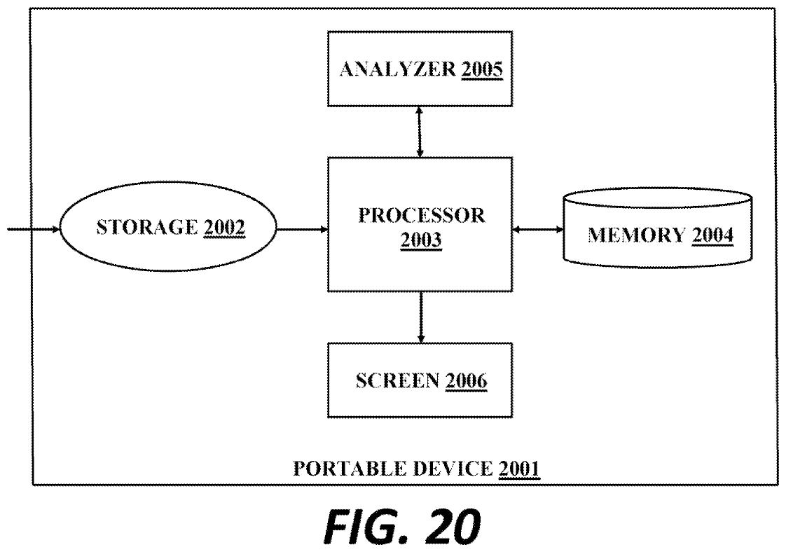

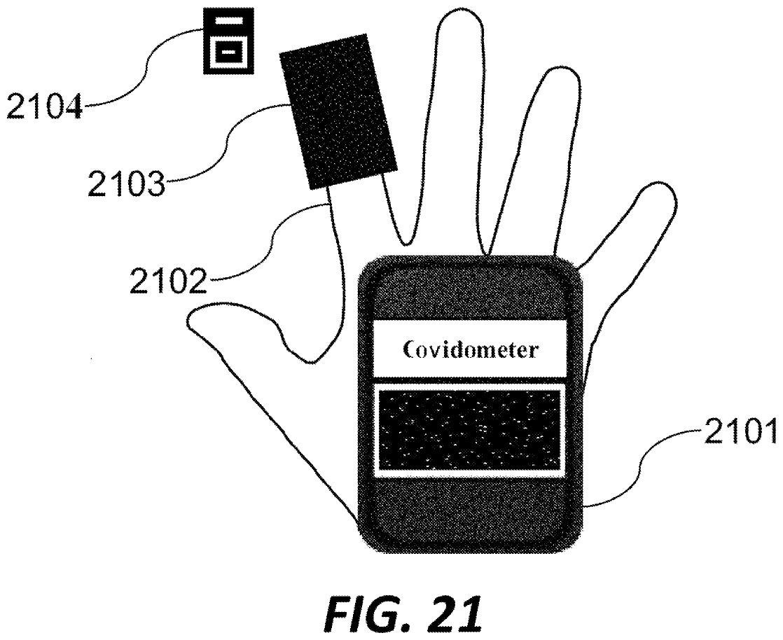

FIGS. 20-21 are diagrams illustrating hardware components and an example of the operation of the device of the “Covidometer”. The system for detecting COVID variants of the present invention can be implemented in the device of the “Covidometer” that determines if a patient has a viral disease of an original COVID-19 virus strain or new mutated COVID variant and requires no lab work. The “Covidometer” of FIG. 20 is the small, portable battery powered device 2001 with computing resources in the form of a storage 2002 for biological sample (e.g., a box for biological materials), processor 2003, memory 2004, analyzer 2005, screen 2006, and it can determine at home whether a patient has contracted COVID disease.

As shown in FIG. 21, the “Covidometer” 2101 is configured to cooperate with the biological sample 2102 by using biochip or biosensors 2103. Types of biosensors 2103 of the “Covidometer” 2101 include those that use enzymes as a biologically responsive material, whole cell metabolism, ligan debinding and antibody-antigen reaction. The types of biological samples 2102 of a patient that can be used for collection of the symptom data values can include antibodies, whole blood, blood plasma, serum samples, nasopharyngeal swab, throat swabs, deep airway material, saliva, any bodily fluid, skin imprint, etc.

The device of the “Covidometer” 2101 of the present invention will be based on biochip or biosensors 2103 capable of distinguishing IgM and IgG antibodies from each other. The “Covidometer” consists of a set of replaceable biochip or biosensors 2103 and a portable device 2101. The whole sampling process is carried out by the biochip or biosensors 2103 that produce a signal (e.g., electrochemical change to detect presence of antigens) detectable by sensors 2104 (e.g., an electrochemical immunosensors capable of distinguishing IgM and IgG antibodies from each other) installed in the device of the “Covidometer” 2101.

The biochip or biosensors 2103 uses aptamers as biosensitive material, which are short, artificially synthesized pieces of DNA or RNA that specifically interact with the blood sample 2102 of a patient. The aptamers are applied to a biochip 2103 coated with a conductive layer of reduced graphene oxide. When aptamers bind to blood proteins (antibodies) 2102 of patient, they gain or lose an extra electron, and this changes the resistance of the conductive layer. The current passing through it increases or decreases, which is recorded by the sensors 2104 of the portable device 2101.

The principle of operation of the “Covidometer” 2101 is similar to the measurement of blood sugar using a glucometer. To determine at home whether a patient has contracted COVID, the patient needs to drop blood 2102 on the biochip or biosensors 2103. When the biosensitive material of the bio sensors 2103 interacts with a blood sample 2102 of a patient containing excess predetermined threshold values of proteins (antibodies), the electrical conductivity of the biosensors 2103 changes, what will be recorded by the sensors 2104. Thereafter the data gathered by using the sensors 2104 will be analyzed in the “Covidometer” according to the algorithm of FIGS. 3-6 of the present invention executable by the processor in the portable device 2101. After a few minutes, the screen of the “Covidometer” 2101 will display the result indicating a presence or absence of COVID disease in a patient.

All publications, patents, and patent documents referred to in this document are incorporated by reference herein in their entirety, as though individually incorporated by reference. While various embodiments of the present invention have been described above, it should be understood that they have been presented by way of example only, and not limitation, and therefore the examples and embodiments described herein are non-limiting examples.

A person skilled in the relevant art will recognize that other components and configurations may be used without parting from the spirit and scope of the embodiments. Furthermore, the various features of the embodiments described herein may be extracted and/or combined to form new embodiments, and each specific element includes all technical equivalents that operate in a similar manner to accomplish a similar purpose.

In the drawings and the description of the drawings herein, certain terminology is used for convenience only, and is not to be taken as limiting the embodiments of the present invention. References to “one embodiment,” “an embodiment,” etc., may indicate that the embodiment(s) of the invention so described may include a particular feature, structure, or characteristic, but not every embodiment necessarily includes the particular feature, structure, or characteristic.

The terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. Also, in the following claims, the terms “including” and “comprising” are open-ended, that is, a system, device, article, or process that includes elements in addition to those listed after such a term in a claim are still deemed to fall within the scope of that claim.

Having thus described a preferred embodiment, it should be apparent to those skilled in the art that certain advantages of the described method and apparatus have been achieved.

It should also be appreciated that various modifications, adaptations, and alternative embodiments thereof may be made within the scope and spirit of the present invention.

What is claimed is:

1. A method for determining COVID disease, the method performed by a processor, the method comprising:
(a) receiving a plurality of values of data from a person representing their symptoms;
(b) calculating first differentials for the first SARS-CoV-2 virus strain by comparing the values to first predetermined symptom threshold values for the first SARS-CoV-2 virus strain;
wherein the first differentials are negative when the values do not exceed the first predetermined symptom threshold values, and positive when the values exceed the first predetermined symptom threshold values;
(c) using the first differentials for the first SARS-CoV-2 virus strain to detect a second SARS-CoV-2 virus strain with a mutated virus genome code based on a correspondence of its symptoms to the first differentials;
(d) calculating second differentials for the second SARS-CoV-2 virus strain by comparing the values to second predetermined symptom threshold values for the second SARS-CoV-2 virus strain;
wherein the second differentials are negative when the values do not exceed the second predetermined symptom threshold values, and positive when the values exceed the second predetermined symptom threshold values;
(e) creating a superset of the first and second differentials;
(f) analyzing the superset to detect correlations within the superset indicative of relationships between the differentials;
(g) determining that the person has the first SARS-CoV-2 virus strain or the second SARS-CoV-virus strain when at least one detected correlation indicates that the person has contracted the first or second strain; and
(h) in response to a determination of (g), outputting a result indicating a presence or absence of SARS-CoV-2 in a person.

2. The method of claim 1, wherein the values of the data represent biochemical and biophysical data of the person.

3. The method of claim 2, wherein the biochemical and biophysical data represent laboratory analysis data of the person.

4. The method of claim 2, wherein the biochemical and biophysical data are gathered by a plurality of sensors.

5. The method of claim 2, wherein the biochemical and biophysical data are gathered by a plurality of biosensors.

6. The method of claim 2, wherein the biochemical and biophysical data are gathered by a test system for an indication of a viral infectious disease.

7. The method of claim 1, wherein the values of the data represent data gathered from a biological sample from the person.

8. The method of claim 7, wherein the data is gathered from medical testing of the biological sample from the person.

9. The method of claim 7, wherein the data is gathered from a biological sample from the person by using a biochip.

10. The method of claim 7, wherein the data represents antibodies data.

11. The method of claim 7, wherein the biological sample is airway material.

12. The method of claim 7, wherein the biological sample is blood plasma or serum.

13. The method of claim 1, further comprising the step of using the second differentials for the second SARS-CoV-2 virus strain to detect a third SARS-CoV-2 virus strain with a mutated virus genome code based on a correspondence of its symptoms to the second differentials.

14. The method of claim 13, wherein third differentials for the third SARS-CoV-2 virus strain are calculated by comparing the values to third predetermined symptom threshold values for the third SARS-CoV-2 virus strain, wherein the third differentials are negative when the values do not exceed the third predetermined symptom threshold values, and positive when the values exceed the third predetermined symptom threshold values.

15. The method of claim 14, wherein a superset of the first, second and third differentials is created, and wherein correlations within the superset are indicative of a presence of the third SARS-CoV-2 virus strain.

16. The method of claim 1, wherein the SARS-CoV-2 virus strain is detected such that its symptoms correspond to a majority of the differentials.

17. The method of claim 1, wherein the SARS-CoV-2 virus strain is detected such that its symptoms correspond to a minority of the differentials.

18. The method of claim 1, further comprising the step of combining the differentials within the superset into groups based on the differences in the differentials, and wherein the superset is analyzed in step (f) to detect correlations between the groups of the differentials.

19. The method of claim 1, wherein the analysis is a combinatorial data analysis, and wherein an order of the differentials within the superset is used to define different combinations of the differentials and their correlations with each other.

20. The method of claim 1, wherein the analysis is a cluster analysis that uses the differences in the differentials within the superset to define multiple groups of the differentials and to find correlations in each group.

21. The method of claim 1, wherein the analysis is a regression analysis that includes constructing a network of curves within the differentials of the superset such that its characteristic figures show correlations between the differentials.

22. The method of claim 1, further comprising the step of analyzing the detected correlations to define the same or similar correlations and combining these correlations into a group, and wherein the determination in step (g) that the person has the SARS-CoV-2 virus strain occurs when at least one detected group of correlations indicates that the person has contracted the strain.

23. A non-transitory computer-readable medium, which stores at least the instructions that are executed by a processor to induce the system to:
(a) receive a plurality of values of data from a person representing their symptoms;
(b) calculate first differentials for the first SARS-CoV-2 virus strain by comparing the values to first predetermined symptom threshold values for the first SARS-CoV-2 virus strain;
wherein the first differentials are negative when the values do not exceed the first predetermined symptom threshold values, and positive when the values exceed the first predetermined symptom threshold values;
(c) use the first differentials for the first SARS-CoV-2 virus strain to detect a second SARS-CoV-2 virus strain with a mutated virus genome code based on a correspondence of its symptoms to the first differentials;
(d) calculate second differentials for the second SARS-CoV-2 virus strain by comparing the values to the second predetermined symptom threshold values for the second SARS-CoV-2 virus strain;
wherein the second differentials are negative when the values do not exceed the second predetermined symptom threshold values, and positive when the values exceed the second predetermined symptom threshold values;
(e) create a superset of the first and second differentials;
(f) analyze the superset to detect correlations within the superset indicative of relationships between the differentials;
(g) determine that the person has the first SARS-CoV-2 virus strain or the second SARS-CoV-2 virus strain when at least one detected correlation indicates that the person has contracted the first or second strain; and
(h) in response to a determination of (g), output a result indicating a presence or absence of SARS-CoV-2 in a person.

24. A system for determining COVID disease in a person, the system comprising:
a plurality of biosensors that collect biological sample from the person;
a plurality of sensors that gather the plurality of values of data from the biological sample;
a transmission system to transmit data from the bio sensors and sensors;
a processor in communication with the transmission system to collect the data from the biosensors and sensors;
a storage that receives and stores collected biological sample;
a server in communication with the processor that comprise a set of the gathered values of the data and a set of predetermined symptom threshold values for SARS-CoV-2 virus strains;
a software application that is downloadable to and executable by the processor to cause the system to perform the steps of
(a) receiving a plurality of values of data from a person representing their symptoms;
(b) calculating first differentials for the first SARS-CoV-2 virus strain by comparing the values to first predetermined symptom threshold values for the first SARS-CoV-2 virus strain;
wherein the first differentials are negative when the values do not exceed the first predetermined symptom threshold values, and positive when the values exceed the first predetermined symptom threshold values;
(c) using the first differentials for the first SARS-CoV-2 virus strain to detect a second SARS-CoV-2 virus strain with a mutated virus genome code based on a correspondence of its symptoms to the first differentials;
(d) calculating second differentials for the second SARS-CoV-2 virus strain by comparing the values to second predetermined symptom threshold values for the second SARS-CoV-2 virus strain;
wherein the second differentials are negative when the values do not exceed the second predetermined symptom threshold values, and positive when the values exceed the second predetermined symptom threshold values;
(e) creating a superset of the first and second differentials;
(f) analyzing the superset to detect correlations within the superset indicative of relationships between the differentials;
(g) determining that the person has the first SARS-CoV-2 virus strain or the second SARS-CoV-2 virus strain when at least one detected correlation indicates that the person has contracted the first or second strain; and
means for outputting a result indicating a presence or absence of SARS-CoV-2 in a person.
